(19) United States
(12) Patent Application Publication
(10) Pub. No.: US 2007/0032555 A1
(43) Pub. Date: Feb. 8, 2007

(54) Novel aryloxyphenyl-propanamines

(75) Inventor: Roger Tung, Lexington, MA (US)

Correspondence Address: EDWARDS & ANGELL, LLP, P.O. BOX 55874, BOSTON, MA, 02205, US

(73) Assignee: Concert Pharmaceuticals Inc., Lexington, MA (US)

(21) Appl. No.: 11/480,265

(22) Filed: Jun. 30, 2006

Related U.S. Application Data
(60) Provisional application No. 60/696,211, filed on Jul. 1, 2005.

(51) Int. Cl. A61K 31/138; C07C 217/16

(52) U.S. Cl. 514/651; 564/365

(57) ABSTRACT

The present invention relates to a non-radioactive, heavy-atom isotopologue of Compound 1 containing one or more deuterium in place of a hydrogen covalently bound to carbon. The Compound 1 isotopologues of the invention are inhibitors of norepinephrine uptake and possess unique biopharmaceutical and pharmacokinetic properties compared to the corresponding non-isotope containing compounds. They may also be used to accurately determine the concentration of Compound 1 in biological fluids. The invention further provides compositions comprising these heavy-atom Compound 1 isotopologue and methods of treating diseases and conditions linked to reduced neurotransmission of norepinephrine.

### RELATED APPLICATION

This application claims the benefit of U.S. provisional patent application Ser. No. 60/696,211 filed on Jul. 1, 2005.

### TECHNICAL FIELD OF THE INVENTION

The present invention relates to an isotopologue of Compound 1, chemically described variously as ((R)-N-methyl-3-phenyl-3-(o-tolyloxy)propan-1-amine), (R)-(−)-N-methyl-γ-(2-methylphenoxy)benzenepropanamine, and R)-(−)-N-methyl-3-phenyl-3-(2-methylphenoxy)propylamine, and its acceptable acid addition salts, and prodrugs thereof, and salts of said prodrugs, and solvates, hydrates, and/or polymorphs of said compound, salts, prodrugs or prodrug salts, if applicable, containing one or more deuterium in place of a hydrogen covalently attached to a carbon atom. The isotopologues of the invention are inhibitors of norepinephrine uptake and possess unique biopharmaceutical and pharmacokinetic properties compared to the corresponding non-isotope containing compounds. The invention further provides compositions comprising a Compound 1 isotopologue and the use of such compositions in methods of treating diseases and conditions that have been linked to reduced neurotransmission of norepinephrine. The invention also provides methods of using the compounds of this invention to determine metabolic liabilities of the all-light atom species and their extraction efficiencies from biological milieu.

### BACKGROUND OF THE INVENTION

Compound 1, of the formula: 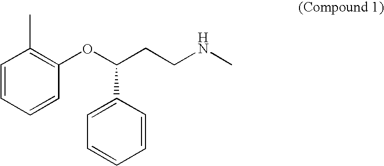
and its pharmaceutically acceptable acid addition salts thereof are disclosed as a useful inhibitors of norepinephrine (NE) uptake. This compound has utility as a psychotropic agent, exhibiting central nervous system activity without significant effect on respiration. It is particularly useful for treating attention-deficit/hyperactivity disorder (ADHD). (Molloy B B and Schmigel K K, U.S. Pat. No. 4,314,081 assigned to Eli Lilly; Heiligenstein J H and Tollefson G D, U.S. Pat. No. 5,658,590 assigned to Eli Lilly). Compound 1 has received approval by the US Food and Drug Administration for the treatment of ADHD and is the first medication to be labeled for treatment of this condition in adults. (Strattera product label dated Feb. 01, 2005. See: http://www.strattera.com/1 6 hcp/1 6 1 prescribing.isp).

Compound 1 is also useful for the treatment of psoriasis and urinary incontinence (Holly R, U.S. Pat. No. 6,683,114 to Eli Lilly; Foreman M M, U.S. Pat. No. 5,441,985 to Eli Lilly). Its application to the treatment of tic disorders; cognitive failure such as from dementia, delerium and schizophrenia; chronic pain including neuropathic pain; stuttering or other communication disorders; pervasive developmental disorders such as autistic disorder and Asperger's disorder; learning disabilities or motor skills disorders; anxiety disorders, especially obsessive-compulsive disorder, including those co-morbid with deficit hyperactivity disorder; cognitive disorders, including those induced by medications; and vasomotor symptoms; have been disclosed (Allen A J and Michelson D, World patent application WO03047560, Lilly applicant; Bymaster F P et. al., World patent application WO03049724, Lilly applicant; Robertson D W, World patent application WO03011272, Upjohn applicant; Kelsey D K, World patent application WO2005021095, Lilly applicant; Allen A J and Kelsey D K, World patent application WO2005020976, Lilly applicant; Sumner C R, World patent application WO2005020975, Lilly applicant; Thomasson H R and Michelson D, World patent application WO0240006, Lilly applicant; Shapira N A et. al., US patent application 20050096349, University of Florida applicant; Deecher D C et. al., US patent application 20040152710). Each of the aforementioned patents and patent applications, and all other patents and patent applications cited in this application, are incorporated herein by reference.

Combinations with additional agents have been disclosed to further extend Compound 1's utility in the treatment or prevention of: pain, inflammation and CNS disorders; ADHD; vasomotor symptoms; nervous system disorders such as childhood disorders, substance disorders, schizophrenia, psychotic disorders and mood disorders, sexual disorders, eating disorders, sleep disorders; depression and/or anxiety; any one or more of depression, anxiety disorders, phobias, avoidant personality disorder, eating disorders, chemical dependencies, Parkinson's diseases, obsessive-compulsive disorder, negative symptoms of schizophrenia, premenstrual syndrome, headache; cognitive failure; obesity; functional bowel disorders; and major psycotic disorders such as schizophreniform disorder, severe schizoaffective disorder with psychotic features, bipolar I disorders with a single manic episode, severe bipolar I disorders with psychotic features, major depressive disorders manifesting a single episode, severe major depressive disorders with psychotic features, severe bipolar I disorders with psychotic features, paranoid personality disorders, major depressive disorders with recurring episodes, and psychotic disorders due to specific general medical conditions. See: Arneric S P, World patent application WO2004060366, Pharmacia applicant; Groppi V E JR et. al., World patent application WO2004060366, Pharmacia applicant; Deecher D C, US patent application 20050130987, Wyeth applicant; Suffin S C et. al., US patent application 20050118286, CNS Response applicant; Arneric S P et. al. US patent application 20050059654, Warner Lambert applicant; Marek G J et. al., US patent application 20050014848, Pfizer applicant; Bymaster F P et. al. US patent application 20050009925, Lilly applicant; Gadde K M and Kaishnan K R R, US patent application 20040198668, Duke University applicant; Landau S B., US patent application 20040147509, Dynogen applicant; and Pickar D et. al. US patent application 20040127489.

Compound 1 has been characterized in rodent models as reducing extracellular levels of norepinephrine, selectively inhibiting presynaptic norepinephrine transporters with limited activity for other central receptors (Bymaster F P et. al., Neuropsychopharmacology 2002 27: 699; Gerlert D R et. al., Neurosci. Lett. 1993 157 203; Wong D T et. al., J. Pharm. Exp. Ther. 1982 222: 61; Corman S L et. al., Am. J. Health Syst. Pharm. 2004 61: 2391).

In human studies, Compound 1 effectively treats ADHD in both children and adults with generally mild to moderate adverse effects. In longer term studies, Compound 1 prevents relapse of ADHD symptoms. Christman, A K et. al. Pharmacotherapy 2004 24: 1020; Simpson D and Plosker G L, Drugs 2004 64: 205. Its use has not been associated with abuse or dependence and it is not a controlled substance.

In a 6-hydroxydopamine-lesion animal model of ADHD, rats treated with Compound 1 demonstrate reductions in their motor hyperactivity without drug-induced stimulation, consistent with human clinical experience. Moran-Gates T et. al., Int. J. Neuropsychopharmacol. 2005 (Epub).

Following oral administration to humans, Compound 1 is well absorbed, followed by extensive oxidative and phase II metabolism with only a minor amount of Compound 1 being excreted unchanged (Sauer J-M et. al., Drug Metab. Dispos. 2003 31: 98). In most of the human population, the major metabolic pathway is rapid hydroxylation at the 4-position of the (2-methyl)phenoxy ring, mediated by cytochrome 2D6 (CYP2D6), followed by glucoronidation and urinary excretion. Oxidative metabolism also occurs to a lesser extent at other positions including the benzylic 2-methyl group and N-methyl group, resulting in demethylation, and possibly other ring oxidation sites.

CYP2D6 is the source of substantial variability in the pharmacokinetics of a number of drugs due to well-known polymorphisms resulting in low CYP2D6 activity in a substantial percentage of the population, including about 2% of Asians and 7-8% of Caucasians (Wolf C R and Smith G, IARC Sci. Publ. 1999 148: 209 (chapter 18); Mura C et. al. Br. J. Clin. Pharmacol. 1993 35: 161; Shimizu T et. al., Drug Metab. Pharmacokinet. 2003 18: 48). In healthy normal volunteers with the predominant allelic polymorphism of CYP2D6, indicated as extensive metabolizers (EM), Compound 1 is 4-hydroxylated predominantly by CYP2D6 and cleared with a mean half life of 18 hours (Sauer J-M et. al., Drug Metab. Dispos. 2003 31: 98). In contrast, in patients with CYP2D6 alleles conferring poor activity of that enzyme, or poor metabolisers (PM), half life is increased to 62 h and total Compound 1 exposure is increased by about 8-10 fold, measured as AUC_{0-τ},. In PM, 4-hydroxylation is mediated by other cytochrome P450 enzymes although at a substantially lower rate (Ring B J et. al., Drug Metab. Dispos. 2002 30: 319), and other oxidized sites are observed in greater abundance. Inhibiting CYP2D6 activity chemically in healthy EMs causes the pharmacokinetics of Compound 1 to resemble that of PMs (Belle D J et. al., J. Clin. Pharmacol. 2002 42: 1219).

Compound 1 was recently reported to be linked to rare but very severe hepatotoxicity in humans (FDA Talk Paper T04-60, Dec. 17, 2004; http://www.fda.gov/bbs/topics/ANSWERS/2004/ANS01335.html).

It is therefore desirable to create a compound displaying the beneficial activities of Compound 1, but with a reduced rate of oxidative metabolism in general and a more consistent rate of oxidative metabolism throughout the population. It is also desirable to create an Compound 1-like compound that avoids hepatotoxicity responses.

### SUMMARY OF THE INVENTION

The present invention solves the problems set forth above by providing a compound of Formula I: 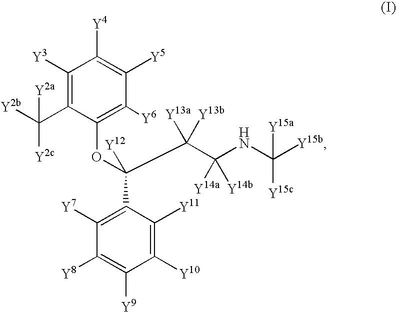
or a prodrug thereof; or a pharmaceutically acceptable salt of said compound or prodrug; or a solvate, hydrate, and/or polymorph of said compound, salt, prodrug or prodrug salt, if applicable, wherein at least one Y is deuterium, and the hydrogen bound to nitrogen is optionally replaced by deuterium, and each carbon is optionally replaced by ¹³C.

Applicant has discovered that the replacement of one or more hydrogen atoms with deuterium in a compound of formula I results in a compound with different and, in certain compounds, superior properties. These compounds, and compositions comprising them, are useful for treating or lessening the severity of disorders characterized by reduced norepinephrine-dependent neurological activity. The compounds and compositions of this invention are also useful as analytical reagents for determining the concentration of the Compound 1 in solution. “Compound 1” as used herein refers to a compound wherein all Y are hydrogen and all carbon atoms are ¹²C, both at their natural isotopic abundance percentages.

The exchange of deuterium in place of hydrogen in an organic molecule typically alters its intrinsic physicochemical properties. This is due to the increased mass of deuterium relative to hydrogen and lowered vibrational frequency of molecular bonds involving deuterium relative to those involving hydrogen (Thornton E R, Ann. Rev. Phys. Chem. 1966 17: 349-372; Halevi, E A et. al. J. Chem. Soc. 1963: 866; Cuma M and Scheiner C, J. Phys. Org. Chem. 1997 10: 383). This change is manifested by numerous physical differences, such as chromatographic behavior, hydrophobicity, hydrogen bond strength, and phase transition temperatures. As an example, deuterium oxide (D₂O, the deuterium analog of water) melts at 3.8° C. in contrast to water, which melts at 0° C., and is more viscous than water. Similarly, hexadeuterated dimethylsulfoxide (DMSO-d₆) melts at a higher temperature, but boils at a lower temperature than its non-deuterated analog. Typically, deuterated compounds will elute faster by reversed-phase HPLC than nondeuterated compounds, apparently due to reduced hydrophobic interactions with the column packing, although the physical chemistry leading to this observed difference is complex (Turowski M et. al., J. Am. Chem. Soc. 2003 125: 13836).

Incorporation of deuterium in place of hydrogen also has significant effects on the physiological and pharmacological activities of the substituted compound. For instance, N-nitrosamines substituted with deuterium can display increased, decreased, or unchanged carcinogenicity depending on where in the compound hydrogen is replaced with deuterium and on the identity of the compound to which substitutions are (Lijinsky W et. al. Food Cosmet. Toxicol. 1982 20: 393; Lijinsky W et. al. JCNI 1982 69: 1127). Similarly, both increases and decreases in bacterial mutagenicity of deuterium-substituted aza-amino acids are known, depending on the identity of the amino acid derivative and position of substitution (Mangold J B et. al. Mutation Res. 1994 308: 33). Reduced hepatotoxicity of certain deuterium-substituted compounds is known (Gordon W P et. al. Drug Metab. Dispos.1987 15: 589; Thompson D C et. al. Chem. Biol. Interact. 1996 101: 1). Deuterium substitution can affect compound's odors (Turin L, Chem. Senses 1996 21: 773) and plasma protein binding (Echmann M L et. al. J. Pharm. Sci. 1962 51: 66; Cherrah Y. et. al. Biomed. Environm. Mass Spectrom. 1987 14: 653; Cherrah Y. et. al. Biochem. Pharmacol. 1988 37: 1311). Changes in the biodistribution and clearance of certain deuterium and ¹³C-substituted compounds suggests changes in their recognition by active transport mechanisms (Zello G A et. al. Metabolism 1994 43: 487; Gately S J et. al. J. Nucl. Med. 1986 27: 388; Wade D, Chem. Biol. Interact. 1999 117: 191).

Replacement of hydrogen with deuterium at sites subject to oxidative metabolism by, for instance, heme proteins such as cytochrome P450 and peroxidase enzymes, is known in certain, but not all, cases to produce a significant reduction in the rate of metabolism due to the primary isotope effect of breaking the C—¹H versus C—²H bond (Guengerich F P et. al. J. Biol. Chem. 2002 277: 33711; Kraus, J A and Guengerich, F P, J. Biol. Chem. 2005 280: 19496; Mitchell K H et. al., Proc. Natl. Acad. Sci. USA 2003 109: 3784; Nelson S D and Trager W F, Drug Metab. Dispos. 2003 31: 1481; Hall L R and Hanzlik, R P J. Biol. Chem. 1990 265: 12349; Okazaki O. and Guengerich F P J. Biol. Chem. 268, 1546; Iwamura S et. al. J. Pharmacobio-Dyn. 1987 10: 229). If the C—H bond breaking step is rate-limiting a substantial isotope effect can be observed. If other steps determine the overall rate of reaction, the isotope effect may be insubstantial. In cases where a rate limiting step of a reaction involves rehybridization of the attached carbon from sp2 to sp3, deuterium substitution often creates a negative isotope effect, speeding up the reaction rate.

Isotope effects caused by substitution of ¹³C for ¹²C can also affect the rate of C—H bond cleavage by enzymatic oxidation. It is further believed that ¹³C substitution combined with deuterium substitution where the two isotopes are bonded to one another (e.g. ¹³C—²H) can be of value due to further stabilizing the C—H bond and thus reducing susceptibility to oxidative metabolism.

¹⁴C-Labeled Compound 1 has been described for use in metabolism studies (Wheeler W J and Kuo F, J. Labelled Compd. Radiopharm. 1995, 36: 213). However the radioactive isotopes ³H and ¹⁴C are physiologically harmful in significant doses and are not useful in routine medicaments.

Although incorporation of stable heavy atoms into specific organic compounds can change their pharmacological properties, general exposure to and incorporation of stable heavy atoms is safe within levels potentially achieved by use of compounds of this invention as medicaments. For instance, the weight percentages of hydrogen and carbon in a mammal (approximately 9% and 18%, respectively) and natural abundances of deuterium and ¹³C (approximately 0.015% and 1.11%, respectively) indicate that a 70 kg human normally contains nearly a gram of deuterium and approximately 140 g of ¹³C. Furthermore, replacement of up to about 15% of normal hydrogen with deuterium has been effected and maintained for a period of days to weeks in mammals, including rodents and dogs, with minimal observed adverse effects (Czajka D M and Finkel A J, Ann. N.Y. Acad. Sci. 1960 84: 770; Thomson J F, Ann. N.Y. Acad. Sci 1960 84: 736; Czakja D M et. al., Am. J. Physiol. 1961 201: 357). Higher deuterium concentrations, usually in excess of 20%, can be toxic in animals. However, brief replacement of as high as 23% of the hydrogen in humans' fluids with deuterium was found not to cause toxicity (Blagojevic N et. al. in “Dosimetry & Treatment Planning for Neutron Capture Therapy”, Zamenhof R, Solares G and Harling O Eds. 1994. Advanced Medical Publishing, Madison Wis. pp.125-134.). In a 70 kg human male, 15% replacement ofthe hydrogen in the fluid compartment with deuterium corresponds to incorporation of approximately 1 kg of deuterium or the equivalent of approximately 5 kg of deuterated water. Replacement of 15% of all of the body's hydrogen with deuterium, as effected in animal studies, would correspond to about twice that amount of deuterium incorporation. These quantities are orders of magnitude beyond the conceived level of administration of any of the deuterium-containing compounds of this invention.

Similarly, replacement of up to 60% of the normally abundant ¹²C with ¹³C has been effected in mice without any observed adverse effects (Gregg C T et. al., Life Sci. 1973 13: 775; Gregg C et. al. in “Proceedings of the Second International conference on Stable Isotopes”, Klein E R and Klein P D Eds. 1975. US Department of Commerce; Springfield Va., pp 64-75). Stable isotope tracers, such as ¹³C-labeled glucose and repeated doses of thousands of milligrams of deuterated water, are used in humans of all ages, including neonates and pregnant women, without reported incident (Pons G and Rey E, Pediatrics 1999 104: 633; Coward W A et. al., Lancet 1979 7: 13; Schwarcz H P, Control. Clin. Trials 1984 5(4 Suppl): 573; Rodewald L E et. al., J. Pediatr. 1989 114: 885; Butte N F et. al. Br. J. Nutr. 1991 65: 3). Thus, it is clear that any stable heavy isotope released, for instance, during the metabolism of compounds of this invention poses no health risk.

Without being bound by theory, applicant believes that the novel compounds of this invention will demonstrate altered and even unexpectedly superior properties as compared to Compound 1. Oxidative metabolism, plasma protein binding, hydrophobicity, hydrogen bond strength, polarizability, and phase transition points are each important parameters contributing to the effectiveness or manufacturability of pharmacological agents. It is predicted that one or more of these altered properties will translate into superior biological, chemical and/or pharmacokinetic properties for a compound of this invention as compared to Compound 1.

Such altered properties include, but are not limited to, higher potency, longer biological half life, increased safety profile, enhanced penetration into the CNS, decreased desolvation energy, enhanced receptor binding affinity, increased physicochemical stability, and enhanced shelf life. It is expected that the compounds of this invention will exhibit one or more of such altered and desirable properties.

It is particularly hypothesized that compounds of this invention will display a reduced rate of oxidative metabolism. This is expected to increase the potency of the compound and reduce norepinephrine uptake on an administered-dose basis in EMs, the majority of the population. It is also expected to reduce the profound difference in pharmacokinetics observed between PMs and EMs. Increased potency will lead to reduced doses required to achieve a desired therapeutic effect, thus decreasing adverse events. Moreover, the compounds of this invention may also reduce or eliminate the severe hepatotoxicity responses seen in rare cases with Compound 1.

Applicant notes that the major primary metabolite of Compound 1 is a para-hydroxy phenyl ether. A similar functional group in troglitazone is believed to sometimes oxidize to a quinone structure followed by protein conjugation, resulting in the rare but severe hepatic dysfunction observed with that drug (He K et. al., Drug Metab. Dispos. 2003 32: 442; Watkins P B and Whitcomb R W, N. Eng. J. Med. 1998 338: 916). Without being bound by theory, applicant hypothesizes that a similar mechanism contributes to Compound 1's hepatotoxicity. Further, applicant believes that slowing the rate of Compound 1 4-hydroxylation in EMs may reduce those patients' exposure to the 4-hydroxylated analog relative to parent drug, and allow more efficient removal of the hydroxylated species by trapping it as the gluceronide, thus avoiding quinone formation and hepatic toxicity.

The altered properties of the compounds of this invention will not, however, obliterate their ability to bind to their receptor targets. This is because such receptor binding is primarily dependent upon non-covalent binding between the receptor and the inhibitor which may be impacted both positively and negatively by isotopic substitution, depending on the specific substitution involved, and any negative effects that a heavy atom of this invention may have on the highly optimized non-covalent binding between compounds of Formula I and norepinephrine uptake proteins will be relatively minor. Major factors contributing to the noncovalent recognition of small molecules by proteins and the binding strength between them include: Van der Waals forces, hydrogen bonds, ionic bonds, molecular reorganization, desolvation energy of the small molecule, hydrophobic interactions and, in certain instances, displacement energy for pre-existing bound ligands. See, for instance, Goodman & Gilman's The Pharmacological Basis of Therapeutics, Tenth Edition, Hardman J G and Limbird L E, eds. McGraw-Hill, 2001.

The compounds of this invention possess molecular topology that is very similar to Compound 1, since exchange of ¹³C for ¹²C is conformationally neutral and exchange of deuterium for hydrogen does not alter molecular shape (Holtzer M E et. al., Biophys. J. 2001 80: 939). Deuterium replacement does cause a slight decrease in Van der Waals radius (Wade D, Chem. Biol. Interact. 1999 117: 191); but applicant believes that such decrease will not greatly reduce binding affinity between the molecule and its receptor. Furthermore, the smaller size of the deuterated compounds prevents their being involved in new undesirable steric clashes with the binding protein relative to the unsubstituted compounds. Neither ¹³C nor deuterium atoms in the compounds of this invention, contribute significantly to hydrogen bonding or ionic interactions with the protein receptors. This is because the major hydrogen bond and ionic interactions formed by the compound with the receptor are through the amine nitrogen and possibly the ether oxygen. Any deuterium atoms attached to the amine nitrogen will be rapidly exchanged with bulk solvent protons under physiological conditions. Protein reorganization or side chain movement will be identical between a compound of this invention and its corresponding light isotopic compounds. As discussed above, desolvation energy of a compound of this invention comprising deuterium is less than that of the corresponding non-deuterium, non-¹³C compound, thus increasing binding affinity for the receptor. Desolvation energy for a compound of this invention comprising ¹³C in place of ¹²C is essentially identical.

Thus, a compound of this invention advantageously retains substantial binding to the norepinephrine uptake proteins and is an active inhibitor of norepinephrine uptake.

### DETAILED DESCRIPTION OF THE INVENTION

The present invention relates to a compound of formula I: 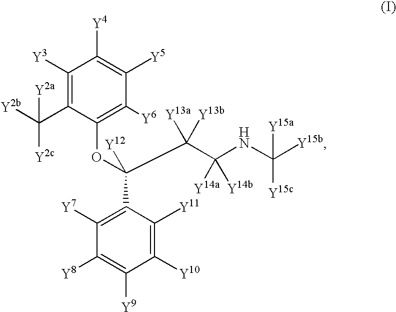
or a prodrug thereof; or a pharmaceutically acceptable salt of said compound or prodrug;
or a solvate, hydrate, and/or polymorph of said compound, salt, prodrug or prodrug salt;
or a pharmaceutically acceptable acid addition salt thereof, wherein:
each Y is independently selected from H or deuterium;
the hydrogen bound to nitrogen is optionally replaced by deuterium;
each carbon atom is optionally replaced with ¹³C; and
wherein at least one Y is deuterium.

According to a preferred embodiment, at least one of Y^{2a}, Y^{2b}, Y^{2c}Y⁴, Y^{15a}, Y^{15b}, or Y^{15c} is deuterium. More preferred is a compound of formula I wherein Y⁴ is deuterium. Yet more preferred is a compound of formula I wherein each of Y³, Y⁴, and Y⁵ are deuterium.

The compounds of the present invention possess an asymmetric carbon. As such, the compounds can exist as the individual stereoisomers as well as the racemic mixture. Accordingly, the compounds of the present invention will include not only the d1-racemates, but also their respective optically active d- and 1-isomers substantially isolated from one another. A “substantially isolated” isomer is one that is predominantly one form relative to other stereoisomers in a combination of stereoisomers. In embodiments, the substantially isolated isomer comprises less than 25% of other stereoisomers, preferably less than 10% of other stereoisomers, more preferably less than 5% of other stereoisomers and most preferably less than 2% of other stereoisomers. Methods of isolating isosteromers from each other are well known in the art.

According to another preferred embodiment, more preferred compounds of formula I are those represented, independently, by Compound 2 and formulas II, III, IV, V, VI, VII, VIII, and IX: 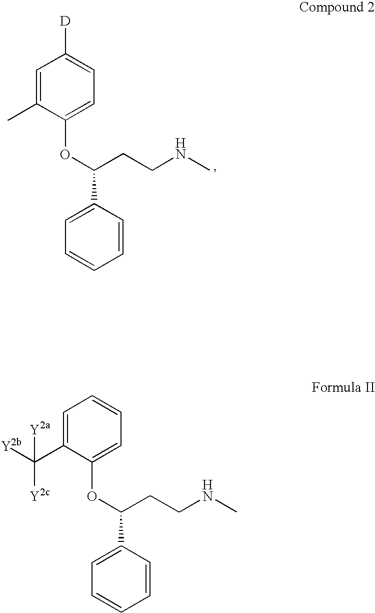 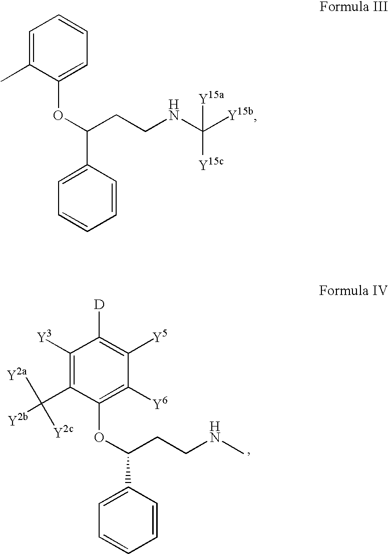 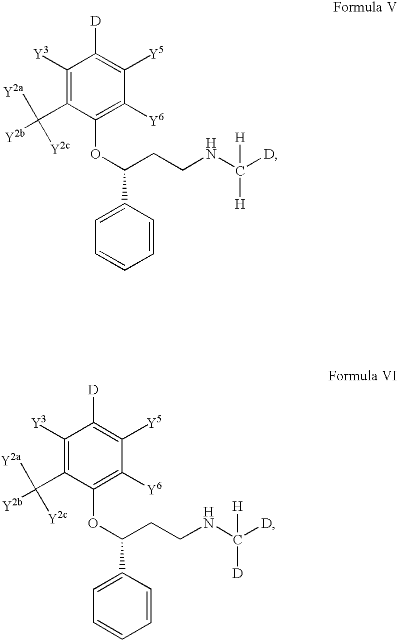 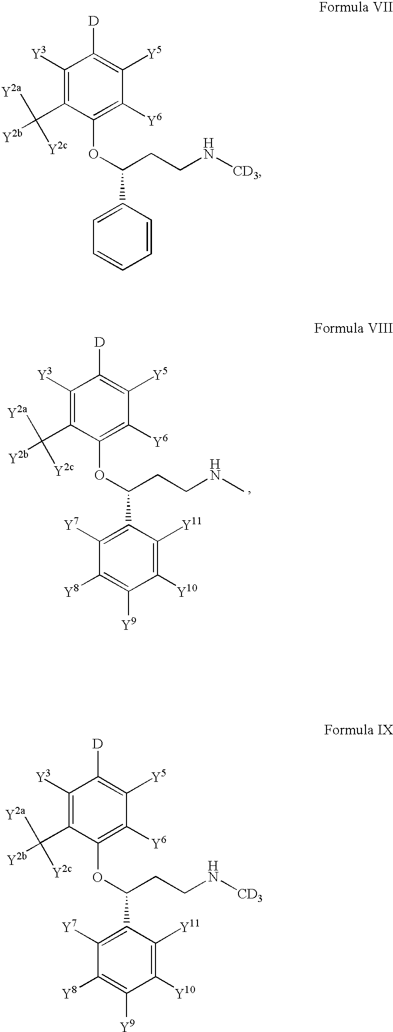
wherein each Y is independently H or D, and wherein in each compound the exchangeable H shown attached to N is optionally replaced with deuterium; and one or more carbons are optionally replaced by with ¹³C. Throughout this specification, reference to “each Y” includes, independently, all “Y” groups including for example Y², Y^{2a}, Y^{2b}, Y^{2c}, Y³, Y⁴, Y⁵, Y⁶, Y⁷, Y⁸, Y⁹, Y^{9a}, Y^{9b}, Y^{9c}, Y¹⁰, Y^{10a}, Y^{10b}, Y¹¹, Y^{11a}, Y^{11b}, Y¹², Y¹³, Y^{13a}, Y^{13b}, Y¹⁴, Y^{14a}, Y^{14b}, Y¹⁵, Y^{15a}, Y^{15b}, Y^{15c}, Y¹⁶, Y^{16a}, Y^{16b}, Y^{16c}, where applicable.

Another aspect of the invention is a compound of any of the formulae herein for use in the treatment or prevention in a subject of a disease, disorder or symptom thereof delineated herein. Another aspect of the invention is use of a compound of any of the formulae wherein in the manufacture of a medicament for treatment or prevention in a subject of a disease, disorder or symptom thereof delineated herein.

Preferred compounds of each of compounds of formulae II-IX are set forth in the tables below. In those tables, D represents deuterium; the exchangeable H shown attached to N is optionally replaced by deuterium; and in each of the compounds one or more carbons are optionally replaced by with ¹³C. An open position in the table is indicative of an “H” or hydrogen atom at that position in the compound.

TABLE 1
Preferred Compounds of Formula II.

| Compound | Y^{2a} | Y^{2b} | Y^{2c} |
|---|---|---|---|
| 3 | D | | |
| 4 | D | D | |
| 5 | D | D | D |

TABLE 2
Preferred Compounds of Formula III.

| Compound | Y^{15a} | Y^{15b} | Y^{15c} |
|---|---|---|---|
| 6 | D | | |
| 7 | D | D | |
| 8 | D | D | D |

TABLE 3
Preferred Compounds of Formula IV.

| Compound | Y^{2a} | Y^{2b} | Y^{2c} | Y³ | Y⁵ | Y⁶ |
|---|---|---|---|---|---|---|
| 9 | | | | | | |
| 10 | | | | D | | |
| 11 | | | | | D | |
| 12 | | | | | | D |
| 13 | | | | D | D | |
| 14 | | | | D | | D |
| 15 | | | | | D | D |
| 16 | | | | D | D | D |
| 17 | D | | | | | |
| 18 | D | | | D | | |
| 19 | D | | | | D | |
| 20 | D | | | | | D |
| 21 | D | | | D | D | |
| 22 | D | | | D | | D |
| 23 | D | | | | D | D |
| 24 | D | | | D | D | D |
| 25 | D | D | | | | |
| 26 | D | D | | D | | |
| 27 | D | D | | | D | |
| 28 | D | D | | | | D |
| 29 | D | D | | D | D | |
| 30 | D | D | | D | | D |
| 31 | D | D | | | D | D |
| 32 | D | D | | D | D | D |
| 33 | D | D | D | | | |
| 34 | D | D | D | D | | |
| 35 | D | D | D | | D | |
| 36 | D | D | D | | | D |
| 37 | D | D | D | D | D | |
| 38 | D | D | D | D | | D |
| 39 | D | D | D | | D | D |
| 40 | D | D | D | D | D | D |

TABLE 4
Preferred Compounds of Formula V.

| Compound | Y^{2a} | Y^{2b} | Y^{2c} | Y³ | Y⁵ | Y⁶ |
|---|---|---|---|---|---|---|
| 41 | | | | | | |
| 42 | | | | D | | |
| 43 | | | | | D | |
| 44 | | | | | | D |
| 45 | | | | D | D | |
| 46 | | | | D | | D |
| 47 | | | | | D | D |
| 48 | | | | D | D | D |
| 49 | D | | | | | |
| 50 | D | | | D | | |
| 51 | D | | | | D | |
| 52 | D | | | | | D |
| 53 | D | | | D | D | |
| 54 | D | | | D | | D |
| 55 | D | | | | D | D |
| 56 | D | | | D | D | D |
| 57 | D | D | | | | |
| 58 | D | D | | D | | |
| 59 | D | D | | | D | |
| 60 | D | D | | | | D |
| 61 | D | D | | D | D | |
| 62 | D | D | | D | | D |
| 63 | D | D | | | D | D |
| 64 | D | D | | D | D | D |
| 65 | D | D | D | | | |
| 66 | D | D | D | D | | |
| 67 | D | D | D | | D | |
| 68 | D | D | D | | | D |
| 69 | D | D | D | D | D | |
| 70 | D | D | D | D | | D |
| 71 | D | D | D | | D | D |
| 72 | D | D | D | D | D | D |

TABLE 5
Preferred Compounds of Formula VI.

| Compound | Y^{2a} | Y^{2b} | Y^{2c} | Y³ | Y⁵ | Y⁶ |
|---|---|---|---|---|---|---|
| 73 | | | | | | |
| 74 | | | | D | | |
| 75 | | | | | D | |
| 76 | | | | | | D |
| 77 | | | | D | D | |
| 78 | | | | D | | D |
| 79 | | | | | D | D |
| 80 | | | | D | D | D |
| 81 | D | | | | | |
| 82 | D | | | D | | |
| 83 | D | | | | D | |
| 84 | D | | | | | D |
| 85 | D | | | D | D | |
| 86 | D | | | D | | D |
| 87 | D | | | | D | D |
| 88 | D | | | D | D | D |
| 89 | D | D | | | | |
| 90 | D | D | | D | | |
| 91 | D | D | | | D | |
| 92 | D | D | | | | D |
| 93 | D | D | | D | D | |
| 94 | D | D | | D | | D |
| 95 | D | D | | | D | D |
| 96 | D | D | | D | D | D |
| 97 | D | D | D | | | |
| 98 | D | D | D | D | | |
| 99 | D | D | D | | D | |
| 100 | | D | D | | | D |
| 101 | D | D | D | D | D | |
| 102 | D | D | D | D | | D |
| 103 | D | D | D | | D | D |
| 104 | D | D | D | D | D | D |

TABLE 6
Preferred Compounds of Formula VII.

| Compound | Y^{2a} | Y^{2b} | Y^{2c} | Y³ | Y⁵ | Y⁶ |
|---|---|---|---|---|---|---|
| 105 | | | | | | |
| 106 | | | | D | | |
| 107 | | | | | D | |
| 108 | | | | | | D |
| 109 | | | | D | D | |
| 110 | | | | D | | D |
| 111 | | | | | D | D |
| 112 | | | | D | D | D |
| 113 | D | | | | | |
| 114 | D | | | D | | |
| 115 | D | | | | D | |
| 116 | D | | | | | D |
| 117 | D | | | D | D | |
| 118 | D | | | D | | D |
| 119 | D | | | | D | D |
| 120 | D | | | D | D | D |
| 121 | D | D | | | | |
| 122 | D | D | | D | | |
| 123 | D | D | | | D | |
| 124 | D | D | | | | D |
| 125 | D | D | | D | D | |
| 126 | D | D | | D | | D |
| 127 | D | D | | | D | D |
| 128 | D | D | | D | D | D |
| 129 | D | D | D | | | |
| 130 | D | D | D | D | | |
| 131 | D | D | D | | D | |
| 132 | D | D | D | | | D |
| 133 | D | D | D | D | D | |
| 134 | D | D | D | D | | D |
| 135 | D | D | D | | D | D |
| 136 | D | D | D | D | D | D |

TABLE 7
Preferred Compounds of Formula VIII.

| Compound | Y^{2a} | Y^{2b} | Y^{2c} | Y³ | Y⁵ | Y⁶ | Y⁷ | Y⁸ | Y⁹ | Y¹⁰ | Y¹¹ |
|---|---|---|---|---|---|---|---|---|---|---|---|
| 137 | | | | | | | | | D | | |
| 138 | | | | D | | | | | D | | |
| 139 | | | | | D | | | | D | | |
| 140 | | | | | | D | | | D | | |
| 141 | | | | D | D | | | | D | | |
| 142 | | | | D | | D | | | D | | |
| 143 | | | | | D | D | | | D | | |
| 144 | | | | D | D | D | | | D | | |
| 145 | D | | | | | | | | D | | |
| 146 | D | | | D | | | | | D | | |
| 147 | D | | | | D | | | | D | | |
| 148 | D | | | | | D | | | D | | |
| 149 | D | | | D | D | | | | D | | |
| 150 | D | | | D | | D | | | D | | |
| 151 | D | | | | D | D | | | D | | |
| 152 | D | | | D | D | D | | | D | | |
| 153 | D | D | | | | | | | D | | |
| 154 | D | D | | D | | | | | D | | |
| 155 | D | D | | | D | | | | D | | |
| 156 | D | D | | | | D | | | D | | |
| 157 | D | D | | D | D | | | | D | | |
| 158 | D | D | | D | | D | | | D | | |
| 159 | D | D | | | D | D | | | D | | |
| 160 | D | D | | D | D | D | | | D | | |
| 161 | D | D | D | | | | | | D | | |
| 162 | D | D | D | D | | | | | D | | |
| 163 | D | D | D | | D | | | | D | | |
| 164 | D | D | D | | | D | | | D | | |
| 165 | D | D | D | D | D | | | | D | | |
| 166 | D | D | D | D | | D | | | D | | |
| 167 | D | D | D | | D | D | | | D | | |
| 168 | D | D | D | D | D | D | | | D | | |
| 169 | | | | | | | | D | | D | |
| 170 | | | | D | | | | D | | D | |
| 171 | | | | | D | | | D | | D | |
| 172 | | | | | | D | | D | | D | |
| 173 | | | | D | D | | | D | | D | |
| 174 | | | | D | | D | | D | | D | |
| 175 | | | | | D | D | | D | | D | |
| 176 | | | | D | D | D | | D | | D | |
| 177 | D | | | | | | | D | | D | |
| 178 | D | | | D | | | | D | | D | |
| 179 | D | | | | D | | | D | | D | |
| 180 | D | | | | | D | | D | | D | |
| 181 | D | | | D | D | | | D | | D | |
| 182 | D | | | D | | D | | D | | D | |
| 183 | D | | | | D | D | | D | | D | |
| 184 | D | | | D | D | D | | D | | D | |
| 185 | D | D | | | | | | D | | D | |
| 186 | D | D | | D | | | | D | | D | |
| 187 | D | D | | | D | | | D | | D | |
| 188 | D | D | | | | D | | D | | D | |
| 189 | D | D | | D | D | | | D | | D | |
| 190 | D | D | | D | | D | | D | | D | |
| 191 | D | D | | | D | D | | D | | D | |
| 192 | D | D | | D | D | D | | D | | D | |
| 193 | D | D | D | | | | | D | | D | |
| 194 | D | D | D | D | | | | D | | D | |
| 195 | D | D | D | | D | | | D | | D | |
| 196 | D | D | D | | | D | | D | | D | |
| 197 | D | D | D | D | D | | | D | | D | |
| 198 | D | D | D | D | | D | | D | | D | |
| 199 | D | D | D | | D | D | | D | | D | |
| 200 | D | D | D | D | D | D | | D | | D | |
| 201 | | | | | | | D | | | | D |
| 202 | | | | D | | | D | | | | D |
| 203 | | | | | D | | D | | | | D |
| 204 | | | | | | D | D | | | | D |
| 205 | | | | D | D | | D | | | | D |
| 206 | | | | D | | D | D | | | | D |
| 207 | | | | | D | D | D | | | | D |
| 208 | | | | D | D | D | D | | | | D |
| 209 | D | | | | | | D | | | | D |
| 210 | D | | | D | | | D | | | | D |
| 211 | D | | | | D | | D | | | | D |
| 212 | D | | | | | D | D | | | | D |
| 213 | D | | | D | D | | D | | | | D |
| 214 | D | | | D | | D | D | | | | D |
| 215 | D | | | | D | D | D | | | | D |
| 216 | D | | | D | D | D | D | | | | D |
| 217 | D | D | | | | | D | | | | D |
| 218 | D | D | | D | | | D | | | | D |
| 219 | D | D | | | D | | D | | | | D |
| 220 | D | D | | | | D | D | | | | D |
| 221 | D | D | | D | D | | D | | | | D |
| 222 | D | D | | D | | D | D | | | | D |
| 223 | D | D | | | D | D | D | | | | D |
| 224 | D | D | | D | D | D | D | | | | D |
| 225 | D | D | D | | | | D | | | | D |
| 226 | D | D | D | D | | | D | | | | D |
| 227 | D | D | D | | D | | D | | | | D |
| 228 | D | D | D | | | D | D | | | | D |
| 229 | D | D | D | D | D | | D | | | | D |
| 230 | D | D | D | D | | D | D | | | | D |
| 231 | D | D | D | | D | D | D | | | | D |
| 232 | D | D | D | D | D | D | D | | | | D |
| 233 | | | | | | | D | D | | | |
| 234 | | | | D | | | D | D | | | |
| 235 | | | | | D | | D | D | | | |
| 236 | | | | | | D | D | D | | | |
| 237 | | | | D | D | | D | D | | | |
| 238 | | | | D | | D | D | D | | | |
| 239 | | | | | D | D | D | D | | | |
| 240 | | | | D | D | D | D | D | | | |
| 241 | D | | | | | | D | D | | | |
| 242 | D | | | D | | | D | D | | | |
| 243 | D | | | | D | | D | D | | | |
| 244 | D | | | | | D | D | D | | | |
| 245 | D | | | D | D | | D | D | | | |
| 246 | D | | | D | | D | D | D | | | |
| 247 | D | | | | D | D | D | D | | | |
| 248 | D | | | D | D | D | D | D | | | |
| 249 | D | D | | | | | D | D | | | |
| 250 | D | D | | D | | | D | D | | | |
| 251 | D | D | | | D | | D | D | | | |
| 252 | D | D | | | | D | D | D | | | |
| 253 | D | D | | D | D | | D | D | | | |
| 254 | D | D | | D | | D | D | D | | | |
| 255 | D | D | | | D | D | D | D | | | |
| 256 | D | D | | D | D | D | D | D | | | |
| 257 | D | D | D | | | | D | D | | | |
| 258 | D | D | D | D | | | D | D | | | |
| 259 | D | D | D | | D | | D | D | | | |
| 260 | D | D | D | | | D | D | D | | | |
| 261 | D | D | D | D | D | | D | D | | | |
| 262 | D | D | D | D | | D | D | D | | | |
| 263 | D | D | D | | D | D | D | D | | | |
| 264 | D | D | D | D | D | D | D | D | | | |
| 265 | | | | | | | | D | D | | |
| 266 | | | | D | | | | D | D | | |
| 267 | | | | | D | | | D | D | | |
| 268 | | | | | | D | | D | D | | |
| 269 | | | | D | D | | | D | D | | |
| 270 | | | | D | | D | | D | D | | |
| 271 | | | | | D | D | | D | D | | |
| 272 | | | | D | D | D | | D | D | | |
| 273 | D | | | | | | | D | D | | |
| 274 | D | | | D | | | | D | D | | |
| 275 | D | | | | D | | | D | D | | |
| 276 | D | | | | | D | | D | D | | |
| 277 | D | | | D | D | | | D | D | | |
| 278 | D | | | D | | D | | D | D | | |
| 279 | D | | | | D | D | | D | D | | |
| 280 | D | | | D | D | D | | D | D | | |
| 281 | D | D | | | | | | D | D | | |
| 282 | D | D | | D | | | | D | D | | |
| 283 | D | D | | | D | | | D | D | | |
| 284 | D | D | | | | D | | D | D | | |
| 285 | D | D | | D | D | | | D | D | | |
| 286 | D | D | | D | | D | | D | D | | |
| 287 | D | D | | | D | D | | D | D | | |
| 288 | D | D | | D | D | D | | D | D | | |
| 289 | D | D | D | | | | | D | D | | |
| 290 | D | D | D | D | | | | D | D | | |
| 291 | D | D | D | | D | | | D | D | | |
| 292 | D | D | D | | | D | | D | D | | |
| 293 | D | D | D | D | D | | | D | D | | |
| 294 | D | D | D | D | | D | | D | D | | |
| 295 | D | D | D | | D | D | | D | D | | |
| 296 | D | D | D | D | D | D | | D | D | | |
| 297 | | | | | | | | D | D | D | |
| 298 | | | | D | | | | D | D | D | |
| 299 | | | | | D | | | D | D | D | |
| 300 | | | | | | D | | D | D | D | |
| 301 | | | | D | D | | | D | D | D | |
| 302 | | | | D | | D | | D | D | D | |
| 303 | | | | | D | D | | D | D | D | |
| 304 | | | | D | D | D | | D | D | D | |
| 305 | D | | | | | | | D | D | D | |
| 306 | D | | | D | | | | D | D | D | |
| 307 | D | | | | D | | | D | D | D | |
| 308 | D | | | | | D | | D | D | D | |
| 309 | D | | | D | D | | | D | D | D | |
| 310 | D | | | D | | D | | D | D | D | |
| 311 | D | | | | D | D | | D | D | D | |
| 312 | D | | | D | D D | | D | D | D | | |
| 313 | D | D | | | | | | D | D | D | |
| 314 | D | D | | D | | | | D | D | D | |
| 315 | D | D | | | D | | | D | D | D | |
| 316 | D | D | | | | D | | D | D | D | |
| 317 | D | D | | D | D | | | D | D | D | |
| 318 | D | D | | D | | D | | D | D | D | |
| 319 | D | D | | | D | D | | D | D | D | |
| 320 | D | D | | D | D | D | | D | D | D | |
| 321 | D | D | D | | | | | D | D | D | |
| 322 | D | D | D | D | | | | D | D | D | |
| 323 | D | D | D | | D | | | D | D | D | |
| 324 | D | D | D | | | D | | D | D | D | |
| 325 | D | D | D | D | D | | | D | D | D | |
| 326 | D | D | D | D | | D | | D | D | D | |
| 327 | D | D | D | | D | D | | D | D | D | |
| 328 | D | D | D | D | D | D | | D | D | D | |
| 329 | | | | | | | D | D | D | D | D |
| 330 | | | | D | | | D | D | D | D | D |
| 331 | | | | | D | | D | D | D | D | D |
| 332 | | | | | | D | D | D | D | D | D |
| 333 | | | | D | D | | D | D | D | D | D |
| 334 | | | | D | | D | D | D | D | D | D |
| 335 | | | | | D | D | D | D | D | D | D |
| 336 | | | | D | D | D | D | D | D | D | D |
| 337 | D | | | | | | D | D | D | D | D |
| 338 | D | | | D | | | D | D | D | D | D |
| 339 | D | | | | D | | D | D | D | D | D |
| 340 | D | | | | | D | D | D | D | D | D |
| 341 | D | | | D | D | | D | D | D | D | D |
| 342 | D | | | D | | D | D | D | D | D | D |
| 343 | D | | | | D | D | D | D | D | D | D |
| 344 | D | | | D | D | D | D | D | D | D | D |
| 345 | D | D | | | | | D | D | D | D | D |
| 346 | D | D | | D | | | D | D | D | D | D |
| 347 | D | D | | | D | | D | D | D | D | D |
| 348 | D | D | | | | D | D | D | D | D | D |
| 349 | D | D | | D | D | | D | D | D | D | D |
| 350 | D | D | | D | | D | D | D | D | D | D |
| 351 | D | D | | | D | D | D | D | D | D | D |
| 352 | D | D | | D | D | D | D | D | D | D | D |
| 353 | D | D | D | | | | D | D | D | D | D |
| 354 | D | D | D | D | | | D | D | D | D | D |
| 355 | D | D | D | | D | | D | D | D | D | D |
| 356 | D | D | D | | | D | D | D | D | D | D |
| 357 | D | D | D | D | D | | D | D | D | D | D |
| 358 | D | D | D | D | | D | D | D | D | D | D |
| 359 | D | D | D | | D | D | D | D | D | D | D |
| 360 | D | D | D | D | D | D | D | D | D | D | D |

TABLE 8
Preferred Compounds of Formula IX.

| Com- | | | | | | | | | | | |
|---|---|---|---|---|---|---|---|---|---|---|---|
| pound | Y^{2a} | Y^{2b} | Y^{2c} | Y³ | Y⁵ | Y⁶ | Y⁷ | Y⁸ | Y⁹ | Y¹⁰ | Y¹¹ |
| 361 | | | | | | | | | D | | |
| 362 | | | | D | | | | | D | | |
| 363 | | | | | D | | | | D | | |
| 364 | | | | | | D | | | D | | |
| 365 | | | | D | D | | | | D | | |
| 366 | | | | D | | D | | | D | | |
| 367 | | | | | D | D | | | D | | |
| 368 | | | | D | D | D | | | D | | |
| 369 | D | | | | | | | | D | | |
| 370 | D | | | D | | | | | D | | |
| 371 | D | | | | D | | | | D | | |
| 372 | D | | | | | D | | | D | | |
| 373 | D | | | D | D | | | | D | | |
| 374 | D | | | D | | D | | | D | | |
| 375 | D | | | | D | D | | | D | | |
| 376 | D | | | D | D | D | | | D | | |
| 377 | D | D | | | | | | | D | | |
| 378 | D | D | | D | | | | | D | | |
| 379 | D | D | | | D | | | | D | | |
| 380 | D | D | | | | D | | | D | | |
| 381 | D | D | | D | D | | | | D | | |
| 382 | D | D | | D | | D | | | D | | |
| 383 | D | D | | | D | D | | | D | | |
| 384 | D | D | | D | D | D | | | D | | |
| 385 | D | D | D | | | | | | D | | |
| 386 | D | D | D | D | | | | | D | | |
| 387 | D | D | D | | D | | | | D | | |
| 388 | D | D | D | | | D | | | D | | |
| 389 | D | D | D | D | D | | | | D | | |
| 390 | D | D | D | D | | D | | | D | | |
| 391 | D | D | D | | D | D | | | D | | |
| 392 | D | D | D | D | D | D | | | D | | |
| 393 | | | | | | | | D | | D | |
| 394 | | | | D | | | | D | | D | |
| 395 | | | | | D | | | D | | D | |
| 396 | | | | | | D | | D | | D | |
| 397 | | | | D | D | | | D | | D | |
| 398 | | | | D | | D | | D | | D | |
| 399 | | | | | D | D | | D | | D | |
| 400 | | | | D | D | D | | D | | D | |
| 401 | D | | | | | | | D | | D | |
| 402 | D | | | D | | | | D | | D | |
| 403 | D | | | | D | | | D | | D | |
| 404 | D | | | | | D | | D | | D | |
| 405 | D | | | D | D | | | D | | D | |
| 406 | D | | | D | | D | | D | | D | |
| 407 | D | | | | D | D | | D | | D | |
| 408 | D | | | D | D | D | | D | | D | |
| 409 | D | D | | | | | | D | | D | |
| 410 | D | D | | D | | | | D | | D | |
| 411 | D | D | | | D | | | D | | D | |
| 412 | D | D | | | | D | | D | | D | |
| 413 | D | D | | D | D | | | D | | D | |
| 414 | D | D | | D | | D | | D | | D | |
| 415 | D | D | | | D | D | | D | | D | |
| 416 | D | D | | D | D | D | | D | | D | |
| 417 | D | D | D | | | | | D | | D | |
| 418 | D | D | D | D | | | | D | | D | |
| 419 | D | D | D | | D | | | D | | D | |
| 420 | D | D | D | | | D | | D | | D | |
| 421 | D | D | D | D | D | | | D | | D | |
| 422 | D | D | D | D | | D | | D | | D | |
| 423 | D | D | D | | D | D | | D | | D | |
| 424 | D | D | D | D | D | D | | D | | D | |
| 425 | | | | | | | D | | | | D |
| 426 | | | | D | | | D | | | | D |
| 427 | | | | | D | | D | | | | D |
| 428 | | | | | | D | D | | | | D |
| 429 | | | | D | D | | D | | | | D |
| 430 | | | | D | | D | D | | | | D |
| 431 | | | | | D | D | D | | | | D |
| 432 | | | | D | D | D | D | | | | D |
| 433 | D | | | | | | D | | | | D |
| 434 | D | | | D | | | D | | | | D |
| 435 | D | | | | D | | D | | | | D |
| 436 | D | | | | | D | D | | | | D |
| 437 | D | | | D | D | | D | | | | D |
| 438 | D | | | D | | D | D | | | | D |
| 439 | D | | | | D | D | D | | | | D |
| 440 | D | | | D | D | D | D | | | | D |
| 441 | D | D | | | | | D | | | | D |
| 442 | D | D | | D | | | D | | | | D |
| 443 | D | D | | | D | | D | | | | D |
| 444 | D | D | | | | D | D | | | | D |
| 445 | D | D | | D | D | | D | | | | D |
| 446 | D | D | | D | | D | D | | | | D |
| 447 | D | D | | | D | D | D | | | | D |
| 448 | D | D | | D | D | D | D | | | | D |
| 449 | D | D | D | | | | D | | | | D |
| 450 | D | D | D | D | | | D | | | | D |
| 451 | D | D | D | | D | | D | | | | D |
| 452 | D | D | D | | | D | D | | | | D |
| 453 | D | D | D | D | D | | D | | | | D |
| 454 | D | D | D | D | | D | D | | | | D |
| 455 | D | D | D | | D | D | D | | | | D |
| 456 | D | D | D | D | D | D | D | | | | D |
| 457 | | | | | | | D | D | | | |
| 458 | | | | D | | | D | D | | | |
| 459 | | | | | D | | D | D | | | |
| 460 | | | | | | D | D | D | | | |
| 461 | | | | D | D | | D | D | | | |
| 462 | | | | D | | D | D | D | | | |
| 463 | | | | | D | D | D | D | | | |
| 464 | | | | D | D | D | D | D | | | |
| 465 | D | | | | | | D | D | | | |
| 466 | D | | | D | | | D | D | | | |
| 467 | D | | | | D | | D | D | | | |
| 468 | D | | | | | D | D | D | | | |
| 469 | D | | | D | D | | D | D | | | |
| 470 | D | | | D | | D | D | D | | | |
| 471 | D | | | | D | D | D | D | | | |
| 472 | D | | | D | D | D | D | D | | | |
| 473 | D | D | | | | | D | D | | | |
| 474 | D | D | | D | | | D | D | | | |
| 475 | D | D | | | D | | D | D | | | |
| 476 | D | D | | | | D | D | D | | | |
| 477 | D | D | | D | D | | D | D | | | |
| 478 | D | D | | D | | D | D | D | | | |
| 479 | D | D | | | D | D | D | D | | | |
| 480 | D | D | | D | D | D | D | D | | | |
| 481 | D | D | D | | | | D | D | | | |
| 482 | D | D | D | D | | | D | D | | | |
| 483 | D | D | D | | D | | D | D | | | |
| 484 | D | D | D | | | D | D | D | | | |
| 485 | D | D | D | D | D | | D | D | | | |
| 486 | D | D | D | D | | D | D | D | | | |
| 487 | D | D | D | | D | D | D | D | | | |
| 488 | D | D | D | D | D | D | D | D | | | |
| 489 | | | | | | | | D | D | | |
| 490 | | | | D | | | | D | D | | |
| 491 | | | | | D | | | D | D | | |
| 492 | | | | | | D | | D | D | | |
| 493 | | | | D | D | | | D | D | | |
| 494 | | | | D | | D | | D | D | | |
| 495 | | | | | D | D | | D | D | | |
| 496 | | | | D | D | D | | D | D | | |
| 497 | D | | | | | | | D | D | | |
| 498 | D | | | D | | | | D | D | | |
| 499 | D | | | | D | | | D | D | | |
| 500 | D | | | | | D | | D | D | | |
| 501 | D | | | D | D | | | D | D | | |
| 502 | D | | | D | | D | | D | D | | |
| 503 | D | | | | D | D | | D | D | | |
| 504 | D | | | D | D | D | | D | D | | |
| 505 | D | D | | | | | | D | D | | |
| 506 | D | D | | D | | | | D | D | | |
| 507 | D | D | | | D | | | D | D | | |
| 508 | D | D | | | | D | | D | D | | |
| 509 | D | D | | D | D | | | D | D | | |
| 510 | D | D | | D | | D | | D | D | | |
| 511 | D | D | | | D | D | | D | D | | |
| 512 | D | D | | D | D | D | | D | D | | |
| 513 | D | D | D | | | | | D | D | | |
| 514 | D | D | D | D | | | | D | D | | |
| 515 | D | D | D | | D | | | D | D | | |
| 516 | D | D | D | | | D | | D | D | | |
| 517 | D | D | D | D | D | | | D | D | | |
| 518 | D | D | D | D | | D | | D | D | | |
| 519 | D | D | D | | D | D | | D | D | | |
| 520 | D | D | D | D | D | D | | D | D | | |
| 521 | | | | | | | | D | D | D | |
| 522 | | | | D | | | | D | D | D | |
| 523 | | | | | D | | | D | D | D | |
| 524 | | | | | | D | | D | D | D | |
| 525 | | | | D | D | | | D | D | D | |
| 526 | | | | D | | D | | D | D | D | |
| 527 | | | | | D | D | | D | D | D | |
| 528 | | | | D | D | D | | D | D | D | |
| 529 | D | | | | | | | D | D | D | |
| 530 | D | | | D | | | | D | D | D | |
| 531 | D | | | | D | | | D | D | D | |
| 532 | D | | | | | D | | D | D | D | |
| 533 | D | | | D | D | | | D | D | D | |
| 534 | D | | | D | | D | | D | D | D | |
| 535 | D | | | | D | D | | D | D | D | |
| 536 | D | | | D | D | D | | D | D | D | |
| 537 | D | D | | | | | | D | D | D | |
| 538 | D | D | | D | | | | D | D | D | |
| 539 | D | D | | | D | | | D | D | D | |
| 540 | D | D | | | | D | | D | D | D | |
| 541 | D | D | | D | D | | | D | D | D | |
| 542 | D | D | | D | | D | | D | D | D | |
| 543 | D | D | | | D | D | | D | D | D | |
| 544 | D | D | | D | D | D | | D | D | D | |
| 545 | D | D | D | | | | | D | D | D | |
| 546 | D | D | D | D | | | | D | D | D | |
| 547 | D | D | D | | D | | | D | D | D | |
| 548 | D | D | D | | | D | | D | D | D | |
| 549 | D | D | D | D | D | | | D | D | D | |
| 550 | D | D | D | D | | D | | D | D | D | |
| 551 | D | D | D | | D | D | | D | D | D | |
| 552 | D | D | D | D | D | D | | D | D | D | |
| 553 | | | | | | | D | D | D | D | D |
| 554 | | | | D | | | D | D | D | D | D |
| 555 | | | | | D | | D | D | D | D | D |
| 556 | | | | | | D | D | D | D | D | D |
| 557 | | | | D | D | | D | D | D | D | D |
| 558 | | | | D | | D | D | D | D | D | D |
| 559 | | | | | D | D | D | D | D | D | D |
| 560 | | | | D | D | D | D | D | D | D | D |
| 561 | D | | | | | | D | D | D | D | D |
| 562 | D | | | D | | | D | D | D | D | D |
| 563 | D | | | | D | | D | D | D | D | D |
| 564 | D | | | | | D | D | D | D | D | D |
| 565 | D | | | D | D | | D | D | D | D | D |
| 566 | D | | | D | | D | D | D | D | D | D |
| 567 | D | | | | D | D | D | D | D | D | D |
| 568 | D | | | D | D | D | D | D | D | D | D |
| 69 | D | D | | | | | D | D | D | D | D |
| 570 | D | D | | D | | | D | D | D | D | D |
| 571 | D | D | | | D | | D | D | D | D | D |
| 572 | D | D | | | | D | D | D | D | D | D |
| 573 | D | D | | D | D | | D | D | D | D | D |
| 574 | D | D | | D | | D | D | D | D | D | D |
| 575 | D | D | | | D | D | D | D | D | D | D |
| 576 | D | D | | D | D | D | D | D | D | D | D |
| 577 | D | D | D | | | | D | D | D | D | D |
| 578 | D | D | D | D | | | D | D | D | D | D |
| 579 | D | D | D | | D | | D | D | D | D | D |
| 580 | D | D | D | | | D | D | D | D | D | D |
| 581 | D | D | D | D | D | | D | D | D | D | D |
| 582 | D | D | D | D | | D | D | D | D | D | D |
| 583 | D | D | D | | D | D | D | D | D | D | D |
| 584 | D | D | D | D | D | D | D | D | D | D | D |

More preferred is a compound selected from any one of Compounds 2 or any one of the compounds set forth in tables 2, 3, 5, or 6. Yet more preferred is a compound selected from any one of Compounds 2, 13, 16, 21, 24, 29, 32, 37, 40, 109, 112, 117, 120, 125, 128, 133, or 136. Even more preferred is a compound selected from any one of Compounds 2, 13, 16, 21, 24, 29, 32, 37, 40, 109, 112, 117, 120, 125, 128, 133, or 136 wherein 0-1 carbon is optionally replaced with ¹³C.

The compounds of the invention may be synthesized by well-known techniques. The starting materials and certain intermediates used in the synthesis of the compounds of this invention are available from commercial sources or may themselves be synthesized using reagents and techniques known in the art, including those synthesis schemes delineated herein. See, for instance, Kjell D P and Lorenz K T, U.S. Pat. No. 6,541,668 to Eli Lilly; Kumar P and Pandley R K, U.S. Pat. No. 6,838,581 to Council of Scientific and Industrial Research; Molloy B B and Schmiegel K K, U.S. Pat. No. 4,314,081 to Eli Lilly; Kumar A et. al., Tetrahedron Lett. 1991 32: 1901; Liu H L et. al. J. Chem. Soc.-Perkin Trans. 1 2000 11: 1767; Ali I S and Sudalai A, Tetrahedron Lett. 2002 43: 5435; Mitchell D and Koenig T M, Synth. Commun. 1995 25: 1231; Gao Y and Sharplesss K B, J. Org. Chem. 1988 53: 4081. Each of these documents is incorporated herein by reference.

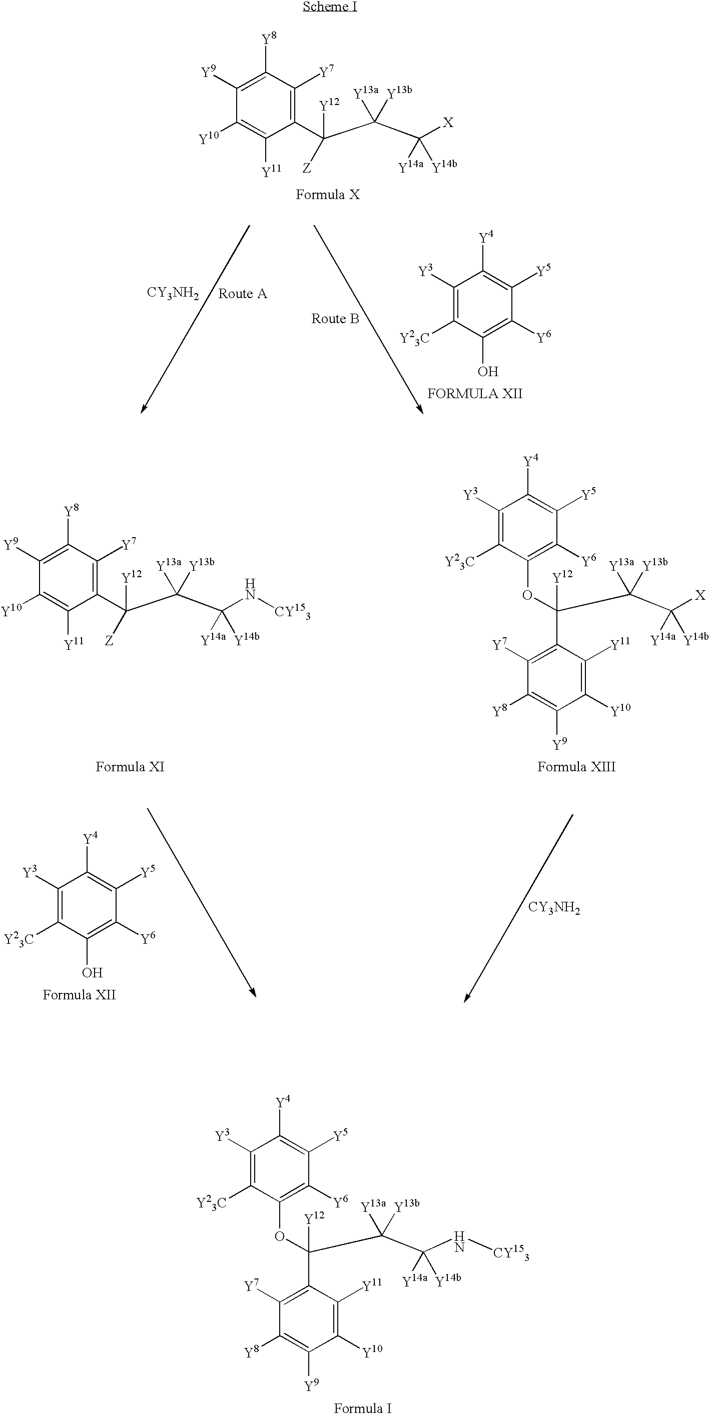

A convenient method for producing compounds of formula I is shown graphically in Scheme I. In Scheme I, X represents a leaving group such as are known in the art, including but not limited to halides such as chloride, bromide and iodide; and sulfonates such as mesylate, tosylate, brosylate, nosylate, triflate, and the like. Other such leaving groups will be apparent to the skilled artesian. Each Y is independently hydrogen or deuterium and each carbon atom is independently ¹²C or ¹³C. Each Z is hydroxyl or X. Each hydrogen attached to nitrogen is optionally deuterium.

As shown in Scheme I, compounds of formula X may be either initially reacted with methylamine or a methylamine synthon, according to Route A, followed by reaction with 2-methylphenol or its deuterated derivatives of formula XII to yield the product of formula I. In Route A, Z is preferably OH. Methylamine synthons are well known in the art and include, for instance, ammonia, azide, carbamates of primary amines such as methyl carbamate, ethyl carbamate, benzyl carbamate and the like, primary amides such as trifluoroacetamide, N,N-dimethylamine, and so forth. Optionally, the compound of formula XI may be N-protected prior to reaction with the compound of formula XII, yielding an N-protected compound of formula I, which is N-deprotected by means known in the art to yield products of formula I, or the compound of formula XI may be N-protected. Suitable N-protecting groups include methyl, benzyl, and substituted benzyl; amides; and carbamates such as methyl carbamate, ethyl carbamte, tert-butoxycarbonyl, benzyloxycarbonyl, and allyloxycarbonyl, among others.

Alternatively, according to Route B, initial reaction of compounds of formula X may be effected with compounds of formula XII to yield intermediates of formula XIII, which is then reacted with methylamine or its deuterated analog to yield products of formula I.

It will be appreciated that compounds of formulae I, X, XI, and XIII contain a chiral center at the carbon bearing Y¹². Any of these compounds may be resolved by means known in the art of organic synthesis, or chiral starting materials of formula X may be synthesized. Conveniently, reaction of compounds of formula XII with those of either formulae X or XI may be effected with stereoinversion at the carbon bearing Y¹². For instance, if Z is OH, the reaction can be mediated using a suitable phosphine and azodicarboxylate (see for instance Mitsunobu O, Synthesis 1981, 1). Suitable phosphines include triphenylphosphine and tributylphosphine, among others. Suitable azodicarboxylates include, for instance, diethylazodicarboxylate, diisopropylazodicarboxylate, and dibenzylazodicarboxylate. Other inverting displacement reactions will be known to those of skill in the art of organic synthesis.

Heavy atom isotopologues of formula X can be synthesized by utilizing methods known for the synthesis of that compound, replacing deuterated and/or ¹³C-substituted starting materials for their corresponding non-deuterated, non-¹³C-containing isotopologues. The term “heavy atom isotopologue” as used herein refers to a compound containing at least one deuterium or ¹³C atom. For instance, as shown in Scheme II, reaction of bromobenzene with allyl alcohol in a palladium mediated Heck reaction yields trans-cinnamyl alcohol (Heck R F, Organic React. 1982 27: 345; Cheeseman N et. al. Proc. Natl. Acad. Sci. USA 2004 101: 5396). Alternatively, Horner-Wadsworth-Emmons reaction of stabilized phosphorus ylides derived from 2-phosphonylacetates with benzaldehyde yields trans-cinnamic acids of formula XIV; see for instance Wadsworth Jr W S and Emmons W D, J. Am. Chem. Soc. 1961 83: 1733. Reduction of the acetate group to the corresponding methanol then yields the desired trans-cinnamyl alcohol of formula XV. Numerous other methods for producing cinnamyl alcohol are known in the art. Allylic epoxidation carried out according to Gao and Sharpless (J. Org. Chem. 1988 53: 4081), followed by regioselective hydride addition and derivitization of the primary alcohol yields the product of formula X, shown as examples wherein Z is OH and X is a sulfonate leaving group. In certain cases, for instance when reactions are rate-limited by abstraction of a hydrogen that has been replaced by deuterium, rates of reaction of deuterated substrates may be different from those of their light atom isotopologues, usually slower. Methods for optimizing reaction conditions, if necessary minimizing competing by-products, are within the scope of the skilled synthetic chemist. Reactions can also be accelerated by means known within the art, for instance by modulating reaction solvents and temperatures, or by use of microwave excitation (Kappe C O, Angew. Chem. Int. Ed. Engl. 2004 43: 6250), which may be of particular value in enhancing the reactivity of sterically hindered and/or electronically deactivated reagents. Reaction optimization and scale-up may advantageously utilize computer-controlled microreactors (Jähnisch, K et al, Angew. Chem. Int. Ed. Engl. 2004 43: 406). Additional reaction schemes and protocols may be determined by the skilled artesian by use of commercially available structure-searchable database software, for instance, SciFinder (CAS division of the American Chemical Society) and CrossFire Beilstein (Elsevier MDL).

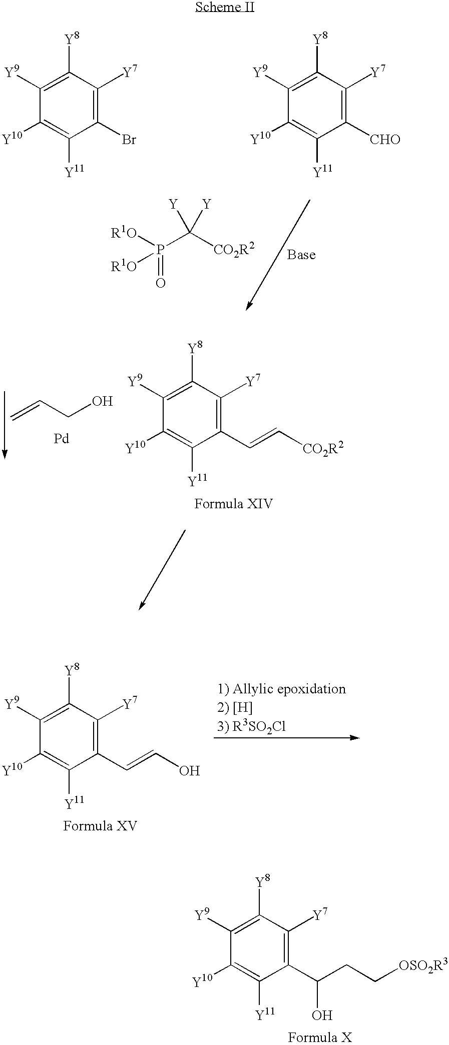

Deuterated and ¹³C-substituted bromobenzene and benzaldehyde isotopologues and their synthetic equivalents (e.g. chlorobenzene) are well known and provide starting materials for many examples of isotopologues of formula X. See for instance C/D/N Isotopes (Pointe-Claire, Quebec, Canada) 2005 catalog; Aldrich Chemicals 2005 catalog; Szele, Helv. Chim. Acta 1981 64: 2733; Bauer G et. al., Chem. Ber. 1976 109: 2231; Frederiksen L B et. al. J. Chem. Res. S 2000 1: 42; Werstiuk; N H and Kadai T, U.S. Pat. No. 3,989,705, Assigned to Canadian Patents and Development; Miura Y et. al., J. Org. Chem. 1997 62: 1188; Korzekwa K R et. al., Biochemistry 1989 28: 9019; Asomaning W A et. al., J. Chem. Soc., Perkin Trans. 1 1973 2: 137. Heavy atom isotopologues of allyl alcohol and haloacetic acids and their esters (precursors to phosphonoacetate ylides used in the above-described Homer-Wadsworth-Emmons reaction) are also commercially available (see for instance C/D/N Isotopes (Pointe-Claire, Quebec, Canada) 2005 catalog; Isotec (Miamisburg, Ohio) 2005 catalog), as are numerous deuterium “hydride” sources. See for instance Kalvin D M and Woodward R D, Tetrahedron 1984 40: 3387 and references therein. Thus, isotopologue positional isomers and combinations of isomers can be readily synthesized.

Heavy atom isotopologues of formula XII are commercially available or may be synthesized by means known in the art. For instance, ring-perdeuterated (Y³⁻⁶=D) and C-perdeuterated (Y²⁻⁶=D) are available from C/D/N Isotopes. Compounds of formula XII may be specifically deuterated at the methyl group (Y²). Applicant uses the term “Y²” to refer to Y^{2a}, Y^{2b} and Y^{2c} collectively. Reductive dehalogenation of 2-methoxybenzyl chloride can be carried out, for instance with RaNi under D₂ gas, with the ate complex of DIBAL-D and n-BuLi, or with SmI₂ in D₂O. See Barrero A F et. al., Synlett 2001 4: 485; Dahlen A et. al., J. Org. Chem. 2003 68: 4870; Kim S and Ahn K H, J. Org. Chem. 1984 49: 1717. Other such reductions will be known to the skilled artesian. Ether cleavage, preferably using aprotic conditions such as, for instance, BBr₃ or trimethylsilyl iodide, yields the product of formula XII wherein one of Y^{2a}, Y^{2b} or Y^{2c} is deuterium (i.e., CY²₃═CH₂D). Scheme III graphically represents these reactions.

Reduction of 2-methoxybenzoic acid or its ester or amide derivatives, for instance with LiAID₄ or DIBAL-D yields dideutero(2-methoxyphenyl)methanol. Removal of the benzylic hydroxyl group can then be effected, for instance, by halogenation using e.g. CCl₄ or CBr₄ in the presence of triphenylphosphine, and followed by reductive dehalogenation as outlined above. If the reduction is carried out using a hydrogen source then the product is dideuterated (two of Y^{2a}, Y^{2b} or Y^{2c} are deuterium; CY²₃═CHD₂), if a deuterium source is used then the product is trideuterated trideuterated (each of Y^{2a}, Y^{2b} and Y^{2c} are deuterium; CY²₃═CD₃). These reactions are shown graphically in Scheme III.

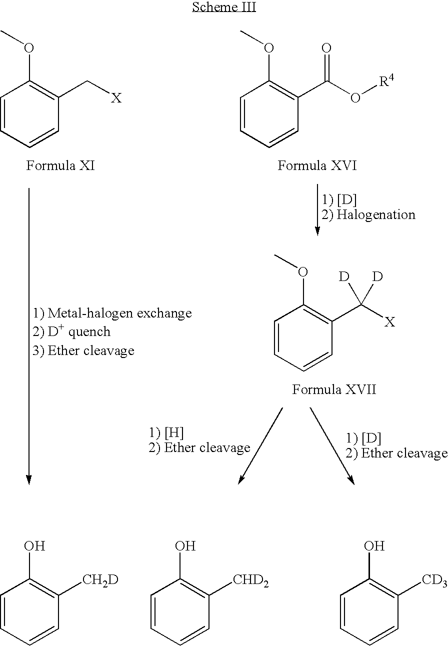

The compound of formula XII wherein Y⁴ is deuterium and all other Y moieties are hydrogen can be synthesized by halogen-metal exchange of commercial 4-bromo-2-methylanisole followed by quenching with a deuterium source, e.g. acetic acid-d or D₂O. See for instance Meyers A I and Mihelich E D, J. Org. Chem. 1975 40: 3158. Similar treatment of other bromo-regioisomers of 2-methylanisole installs deuterium at the other ring positions (see for instance Roberts J C and Rance M J, J. Chem. Soc. Section C 1971 7: 1247; McCowan J R et. al. World patent application WO0136414, Lilly Applicant; Ando M and Emoto S, Bull. Chem. Soc. Jpn. 1978 51: 2435; Ness S et. al. Biochemistry 2000 39: 5312; Gates P S et. al., U.S. Pat. No. 4,263,037 to Fisons). Regioselective reductive dehalogenation of ortho and para-bromophenols can be effected with deuteroiodic acid in deuterium oxide, analogous to the reaction carried out with the protic reagents; see Britlain J M et. al. J. Chem. Soc., Perkin Trans. 1 1984, 32. Ether cleavage as above yields the compound of formula XII.

Further deuteration on the tolyl methyl (Y^{2a-c}) group can be effected on the products of the above deuteration reactions. For instance, benzylic halogenation followed by metal-halogen exchange and deuterium quench, analgous to that shown in Scheme III for compounds of Formula XI, yields further monodeuteration at the methyl group. Suitable reagents for benzylic halogenation include, for instance, N-halosuccinimides and radical initiators such as AIBN or benzoyl peroxide. Alternatively, for instance, oxidation of the methyl group to the level of a carboxylic acid can be carried out, followed by reduction with deuterium as outlined in Scheme III for compounds of formula XVII, yielding the di- and tri-deuterated methyl species of formula XII. Suitable oxidizing agents include, for instance, Jones reagent, or preferably milder conditions using a two-step procedures such as bipyridinium chlorochromate-catalyzed peroxide, IBX (o-iodylbenzoic acid), or lead tetraacetate oxidation, followed by a second oxidation to the carboxylic acid, for instance with KMnO₄. See e.g. Handbook of Reagents for Organic Synthesis, Vol. 3: Oxidizing and Reducing Agents, Burke S D and Danheiser R L (Eds.) 1999 John Wiley & Sons, New York; Rathore R et. al., Synth. Commun. 1986 16: 1493; Nicolau K C et. al. J. Am. Chem. Soc. 2002 124: 2245. Thus, it is clear that deuterium may be incorporated into any position of compounds of formula XII.

Another embodiment is a compound of any of the formulae herein made by a process delineated herein, including the processes exemplified in the schemes and examples herein. The chemicals used in the synthetic routes described herein may include, for example, solvents, reagents, catalysts, and protecting group and deprotecting group reagents. The methods described herein may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3R^{d} Ed., John Wiley and Sons (1999); L. Fieser and M. Fieser, Fieser and Fieser 's Reagents for Organic Synthesis, John Wiley and Sons (1994); and L. Paquette, ed., Encyclopedia of Reagents for Organic Synthesis, John Wiley and Sons (1995) and subsequent editions thereof.

The methods described herein may also additionally include steps, either before or after the steps described specifically herein, to add or remove suitable protecting groups in order to ultimately allow synthesis of the compound of the formulae described herein. The methods delineated herein contemplate converting compounds of one formula to compounds of another formula. The process of converting refers to one or more chemical transformations, which can be performed in situ, or with isolation of intermediate compounds. The transformations can include reacting the starting compounds or intermediates with additional reagents using techniques and protocols known in the art, including those in the references cited herein. Intermediates can be used with or without purification (e.g., filtration, distillation, crystallization, chromatography).

According to another embodiment, the invention provides any of above-described intermediate compounds X, XI, or XIII comprising at least one deuterium atom or at least one ¹³C atom.

Combinations of substituents and variables envisioned by this invention are only those that result in the formation of stable compounds. The term “stable”, as used herein, refers to compounds which possess stability sufficient to allow manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition that has been linked to reduced neurotransmission of norepinephrine).

The compounds of this invention include the compounds themselves, as well as their salts, solvate, hydrate, polymorph, or prodrugs, if applicable. As used herein, the term “pharmaceutically acceptable salt,” is a salt formed from, for example, an acid and a basic group of a compound of any one of the formulae disclosed herein. Acids commonly employed to form such salts include inorganic acids such as hydrochloric, hydrobromic, hydroiodic, sulfuric and phosphoric acid, as well as organic acids such as para-toluenesulfonic, methanesulfonic, oxalic, para-bromophenylsulfonic carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycolate, maleate, tartrate, methanesul fonate, propanesul fonate, naphthalene-1-sulfonate, naphthalene-2-sulfonate, mandelate and the like salts. Preferred pharmaceutically acceptable acid addition salts include those formed with mineral acids such as hydrochloric acid and hydrobromlc acid, and especially those formed with organic acids such as maleic acid.

As used herein, the term “hydrate” means a compound of the present invention or a salt thereof, which further includes a stoichiometric or non-stoichiometric amount of water bound by non-covalent intermolecular forces.

The term “solvate” means a compound of the present invention or a salt thereof, which further includes a stoichiometric or non-stoichiometric amount of solvent such as water, acetone, ethanol, methanol, dichloromethane, 2-propanol, or the like, bound by non-covalent intermolecular forces.

As used herein, the term “polymorph” means solid crystalline forms of a compound of the present invention or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), hygroscopicity, solubility, and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein and unless otherwise indicated, the term “prodrug” means a derivative of a compound that can hydrolyze, oxidize, or otherwise react under biological conditions (in vitro or in vivo) to provide a compound of this invention. Prodrugs may only become active upon such reaction under biological conditions, or they may have activity in their unreacted forms. Examples of prodrugs contemplated in this invention include, but are not limited to, analogs or derivatives of compounds of any one of the formulae disclosed herein that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by Burger's Medicinal Chemistry and Drug Discovery (1995) 172-178, 949-982 (Manfred E. Wolff ed., 5th ed); see also Goodman and Gilman's, The Pharmacological basis of Therapeutics, 8th ed., McGraw-Hill, Int. Ed. 1992, “Biotransformation of Drugs”.

As used herein and unless otherwise indicated, the terms “biohydrolyzable amide”, “biohydrolyzable ester”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as uptake, duration of action, or onset of action; or 2) is itself biologically inactive but is converted in vivo to a biologically active compound. Examples of biohydrolyzable amides include, but are not limited to, lower alkyl amides, α-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, amino acids, hydroxyalkylamines, heterocyclic and heteroaromatic amines, and polyether amines.

The term “isotopologue” refers to species that differ from a compound of this invention only in the isotopic composition of their molecules or ions. The terms “lighter isotopologue” and “lighter atom isotopologue” as used herein, refer to species that differs from a compound of this invention in that it comprises one or more light isotopic atoms ¹H or ¹²C at positions occupied by a deuterium or ¹³C. For the purposes of this invention, ¹¹C is not referred to as a light isotope of carbon. It will be readily apparent that all lighter isotopologues except the lighter isotopologue devoid of heavy isotopic atoms (i.e., Compound 1) are compounds according to this invention. Thus, for example, a compound of formula II, wherein Y², Y³ and Y⁴ are each deuterium has lighter isotopologues wherein Y² and Y³ are deuterium and Y⁴ is hydrogen; Y² and Y⁴ are deuterium and Y³ is hydrogen; Y³ and Y⁴ are deuterium and Y² is hydrogen; Y² is deuterium and Y³ and Y⁴ are hydrogen; Y³ is deuterium and Y² and Y⁴ are hydrogen; Y⁴ is deuterium and Y² and Y³ are hydrogen; and Y², Y³ and Y⁴ are all hydrogen, this latter compound being Compound 1.

Chemical naming terminology can be complex and different chemical names can often reasonably be applied to the same structure. To avoid any confusion, “Compound 1” refers to the chemical structure shown herein for that compound.

It will be recognized that many commonly occurring atoms in biological systems exist naturally as mixtures of isotopes. Thus, the corresponding non-deuterated, non-¹³C compounds of the compounds of this invention may inherently comprise small amounts of deuterated and/or ¹³C-containing isotopologues. The present invention excludes such contaminants from its scope in that the term “compound” as used in this invention refers to a composition of matter that is predominantly a specific isotopologue. In embodiments, the “compound” contains less than 10%, preferably less than 5%, and more preferably less than 2% of any other specific isotopologue, including the corresponding non-deuterated, non-¹³C compound. Compositions of matter that may contain greater than 10% of any other specific isotopologue are referred to herein as mixtures and must meet the parameters set forth below.

The term “heavy atom” refers to isotopes of higher atomic weight than the predominant naturally occurring isotope.

The term “stable heavy atom” refers to non-radioactive heavy atoms.

Both “²H” and “D” refer to deuterium.

“AIBN” refers to 2,2′-azo-bis(isobutyronitrile)

“aq.” Refers to aqueous

The invention further provides compositions comprising a mixture of a compound of this invention and its lighter isotopologues. These mixtures may be occur, for instance, simply as the result of an inefficiency of incorporating the isotope at a given position; intentional or inadvertent exchange of protons for deuterium, e.g. exchange of bulk solvent for heteroatom-attached deuterium; or intentional mixtures of pure compounds.

In one embodiment, such mixtures comprise at least about 50% of the heavy atom isotopic compound (i.e., less than about 50% of lighter isotopologues). More preferable is a mixture comprising at least 80% of the heavy atom isotopic compound. Even more preferable is a mixture comprising at least 90% of the heavy atom isotopic compound. Even more preferable is a mixture comprising at least 95% of the heavy atom isotopic compound. Most preferred is a mixture comprising at least 98% of the heavy atom isotopic compound.

In an alternate embodiment the mixture comprises a compound and its lighter isotopologues in relative proportions such that at least about 50%, preferably at least 80%, more preferably at least 90%, even more preferably at least 95% and most preferably at least 98% of the compounds in said mixture comprise an isotope at each position containing a stable heavy atom isotope in the full isotopic compound. The following exemplifies this definition. A hypothetical compound of the invention contains deuterium at positions Y³, Y⁴ and Y⁵. A mixture comprising this compound and all of its potential lighter isotopologues and the relative proportion of each is set forth in the table below.

TABLE 9

| | | | | Relative |
|---|---|---|---|---|
| | Y³ | Y⁴ | Y⁵ | Amt |
| Compound | D | D | D | 40% |
| Isotopologue 1 | D | D | H | 15% |
| Isotopologue 2 | D | H | D | 15% |
| Isotopologue 3 | H | D | D | 15% |
| Isotopologue 4 | D | H | H | 4% |
| Isotopologue 5 | H | D | H | 4% |
| Isotopologue 6 | H | H | D | 4% |
| Isotopologue 7 | H | H | H | 3% |
| % of | (40% + 15% + 15% + 4%) = | 74% | 74% | |
| compounds | 74% | | | |
| comprising an | | | | |
| isotope at | | | | |
| position | | | | |

From the table it can be seen that the compound plus lighter isotopologues 1, 2 and 4 comprises the isotope deuterium at position Y³. These compounds are present in the mixture at relevant amounts of 40%, 15%, 15% and 4%. Thus, 74% of the mixture comprises the isotope at Y³ that is present in the compound.

The invention also provides compositions comprising an effective amount of a compound of this invention (e.g., including any of the formulae herein) or a pharmaceutically acceptable salt, solvate, hydrate, polymorph or prodrug, if applicable, of said compound or said intermediate; and an acceptable carrier. Preferably, a composition of this invention is formulated for pharmaceutical use (“a pharmaceutical composition”), wherein the carrier is a pharmaceutically acceptable carrier. The carrier(s) must be “acceptable” in the sense of being compatible with the other ingredients of the formulation and, in the case of a pharmaceutically acceptable carrier, not deleterious to the recipient thereof in amounts typically used in medicaments.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets and sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers or both, and then if necessary shaping the product.

In certain preferred embodiments, the compound is administered orally. Compositions of the present invention suitable for oral administration may be presented as discrete units such as capsules, sachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein. Methods of formulating such slow or controlled release compositions of pharmaceutically active ingredients, such as those herein and other compounds known in the art, are known in the art and described in several issued U.S. patents, some of which include, but are not limited to, U.S. Pat. Nos. 4,369,172; and 4,842,866, and references cited therein. Coatings can be used for delivery of compounds to the intestine (see, e.g., U.S. Pat. Nos. 6,638,534, 5,217,720, and 6,569,457, 6,461,631, 6,528,080, 6,800,663, and references cited therein).

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added. Surfactants such as sodium lauryl sulfate may be useful to enhance dissolution and absorption.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches and iontophoretic administration are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or central nervous system) relative to the parent species. Preferred prodrugs include derivatives where a group that enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development; 2 ed.; Overseas Publ.: Amsterdam, 1996; pp 351-385; Pitman, I. H. Medicinal Research Reviews 1981, 1, 189-214.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released form said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

The present invention further provides pharmaceutical compositions comprising an effective amount of one or more compound of the invention, or a salt, solvate, hydrate, polymorph, or prodrug of said compound or intermediate in combination with an effective amount of another therapeutic agent useful for treating or preventing pain, inflammation and CNS disorders; ADHD; vasomotor symptoms; nervous system disorders such as childhood disorders, substance disorders, schizophrenia, psychotic disorders and mood disorders, sexual disorders, eating disorders, sleep disorders; depression and/or anxiety; any one or more of depression, anxiety disorders, phobias, avoidant personality disorder, eating disorders, chemical dependencies, Parkinson's diseases, obsessive-compulsive disorder, negative symptoms of schizophrenia, premenstrual syndrome, headache; cognitive failure; obesity; functional bowel disorders; and major psychotic disorders such as schizophreniform disorder, severe schizoaffective disorder with psychotic features, bipolar I disorders with a single manic episode, severe bipolar I disorders with psychotic features, major depressive disorders manifesting a single episode, severe major depressive disorders with psychotic features, severe bipolar I disorders with psychotic features, paranoid personality disorders, major depressive disorders with recurring episodes, and psychotic disorders due to specific general medical conditions.

Such other therapeutic agents useful in combination with the compounds of this invention include, but are not limited to, a cyclooxygenase-2 selective inhibitor; an alpha7 nAChR full agonist; a selective adrenergic α_{2B} receptor antagonist; an adrenergic beta-blocker; a CaV2.2 alpha-2-delta (A2D) ligand or a prodrug thereof, or a pharmaceutically acceptable salt of said A2D ligand or said prodrug; a serotonin uptake inhibitor; an additional norepinephrine uptake inhibitor; a weight-loss promoting anticonvulsant; a 5-HT₃ receptor antagonists; or an atypical D₂ antagonist.

Examples of cyclooxygenase-2 selective inhibitor include celecoxib, JTE-522, deracoxib, parecoxib, valdecoxib, etoricoxib, rofecoxib,N-(2-cyclohexyloxynitrophenyl)methane sulfonamide, COX189, ABT963, meloxicam, BMS-347070, prodrugs of any of the foregoing, and mixtures thereof.

Examples of alpha7 nAChR full agonists include those disclosed in PCT International patent application WO2004052461.

Examples of selective adrenergic α_{2B} receptor antagonists include 2-(1-ethyl-2-imidazoyl)methyl-1,4-benzodioxan(imiloxan), 2-[(2,3-dihydro-1,4-benzodioxin-2-yl)methyl]-1-ethyl-1H-imidazole, 2-[2-[4-(2-methoxyphenyl)-1-piperazinyl]ethyl]-4,4-dimethyl-1,3(2H,4H)-isoquinolinedione (ARC 239), a pharmaceutically acceptable salt of any of the above, or a combination of two or more of the above.

Examples of adrenergic beta-blockers include prenalteraol, xamoterol, propranolol, atenolol, betaxolol, nadolol, carvedilol, sotolol, timolol, labetolol, acebutolol, pindolol, esmolol, metoprolol, bisoporol and bucindolol.

Examples of CaV2.2 alpha-2-delta (A2D) ligands include gabapentin and pregabalin.

Examples of serotonin uptake inhibitors include femoxetine, fluoxetine, fluvoxamine, indalpine, indeloxazine, milnacipran, paroxetine, sertraline, sibutramine, zimeldine, citalopram, escitalopram, fenfluramine, venlafaxine, and duloxetine and those disclosed in United States Patent Application 20050014848.

Examples of additional norepinephrine uptake inhibitors include those disclosed in U.S. Pat. No. 5,281,624.

Examples of weight loss-inducing anticonvulsants include zonisamide and topiramate.

Examples of 5-HT₃ receptor antagonists include indisetron, YM-114 ((R)-2,3-dihydro-1-[(4,5,6,7-tetrahydro-1H-benzimidazol-5-yl-)carbonyl]-1-H-indole), granisetron, talipexole, azasetron, bemesetron, tropisetron, ramosetron, ondansetron, palonosetron, lerisetron, alosetron, N-3389, zacopride, cilansetron, E-3620 ([3(S)-endo]-4-amino-5-chloro-N-(8-methyl-8-azabicyclo[3.2.1-]oct-3-yl-2[(1-methyl-2-butynyl)oxy]benzamide), lintopride, KAE-393, itasetron, zatosetron, dolasetron, zacopride, both separated stereoisomers and stereoisomeric mixtures, renzapride both separated stereoisomers and stereoisomeric mixtures, (−)-YM-060, DAU-6236, BIMU-8 and GK-128 [2-[2-methylimidazol-1-yl)methyl]-benzothiochromen-1-one monohydrochloride hemihydrate].

Examples of atypical D₂ antagonists include olanzapine, quetiapine, risperidone, sertindole and ziprasidone.

In another embodiment, the invention provides separate dosage forms of a compound of this invention and a second therapeutic agent that are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of a disorder associated with reduced neurotransmission of norepinephrine, prevent the advancement of a disorder characterized by reduced interstitial concentrations norepinephrine, cause the regression of a disorder characterized by reduced interstitial concentrations of norepinephrine, or enhance or improve the prophylactic or therapeutic effect(s) of another therapy. In certain preferred embodiments, treatment according to the invention provides a reduction in or prevention of at least one symptom or manifestation of a disorder that has been linked to reduced neurotransmission of norepinephrine, as determined in vivo or in vitro of at least about 10%, more preferably 20%, 30%, 40%, 50%, 60%, 70%, 80%, 90%, 95%, 98% or 99%. With respect to inhibition of norepinephrine uptake, the term “effective amount” means an amount that results in a detectable increase in the amount or concentration norepinephrine in a patient or in a biological sample, the correction of or relief from a behavior, deficit, symptom, syndrome or disease that has been linked to reduced neurotransmission norepinephrine, alone or in combination with another agent or agents; or the induction of a behavior, activity or response that has been linked to normalized or increased neurotransmission of norepinephrine.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardley, N.Y., 1970, 537. An effective amount of a compound of this invention can range from about 0.001 mg/kg to about 500 mg/kg, more preferably 0.01 mg/kg to about 50 mg/kg, more preferably 0.1 mg/kg to about 2.5 mg/kg. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

For pharmaceutical compositions that comprise additional therapeutic agents, an effective amount of the other agent is between about 20% and 100% of the dosage normally utilized in a monotherapy regime using just that additional agent. Preferably, an effective amount is between about 70% and 100% of the normal monotherapeutic dose. The normal monotherapeutic dosages of these additional therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

It is expected that some of the additional therapeutic agents listed above will act synergistically with the compounds of this invention. When this occurs, its will allow the effective dosage of the additional therapeutic agent and/or the compound of this invention to be reduced from that required in a monotherapy. This has the advantage of minimizing toxic side effects of either the additional therapeutic agent of a compound of this invention, synergistic improvements in efficacy, improved ease of administration or use and/or reduced overall expense of compound preparation or formulation.

Methods of Treatment

In one embodiment, the present invention provides a method of inhibiting the uptake of norepinephrine in a subject comprising the step of administering to said subject an effective amount of a compound of this invention, preferably combined in a composition with a pharmaceutically acceptable carrier. Preferably the method is employed to treat a subject suffering from or susceptible to one or more disease or disorder selected from ADHD; psoriasis; urinary incontinence; tic disorders, particularly those co-morbid with ADHD; cognitive failure such as from dementia, delirium and schizophrenia; chronic pain including neuropathic pain; stuttering or other communication disorders; pervasive developmental disorders such as autistic disorder and Asperger's disorder; learning disabilities or motor skills disorders; anxiety disorders, especially obsessive-compulsive disorder, including those co-morbid with deficit hyperactivity disorder; cognitive disorders, including those induced by medications; or vasomotor symptoms. Other embodiments include any of the methods herein wherein the subject is identified as in need of the indicated treatment.

In another embodiment, the method of treatment further comprises the step of administering to said patient a second therapeutic agent which alone or in combination with Compound 1 is effective to treat one or more of pain; inflammation; a CNS disorder; ADHD; a vasomotor symptom; a nervous system disorder, such as a childhood disorder, substance disorder, schizophrenia, psychotic disorder, mood disorder, sexual disorder, eating disorder, or sleep disorder; depression; anxiety; a phobia; avoidant personality disorder; a chemical dependency; a Parkinson's disease; obsessive-compulsive disorder;, premenstrual syndrome; headache; cognitive failure; obesity; functional bowel disorder; or a major psychotic disorder, such as schizophreniform disorder, severe schizoaffective disorder with psychotic features, a bipolar I disorder with a single manic episode, a severe bipolar I disorder with psychotic features, a major depressive disorder manifesting a single episode, a severe major depressive disorder with psychotic features, a paranoid personality disorder, a major depressive disorder with recurring episodes, or a psychotic disorder due to specific general medical conditions.

The additional therapeutic agent may be administered together with a compound of this invention as part of a single dosage form or as multiple dosage forms. Alternatively, the additional agent may be administered prior to, consecutively with, or following the administration of a compound of this invention. In such combination therapy treatment, both the compounds of this invention and the other therapeutic agent(s) are administered by conventional methods. The administering of the other therapeutic agent may occur before, concurrently with, and/or after the administering of the compound of this invention. When the administering of the other therapeutic agent occurs concurrently with a compound of this invention, the two (or more) agents may be administered in a single dosage form (such as a composition of this invention comprising a compound of the invention and an additional therapeutic agent as described above), or in separate dosage forms. The administration of a composition of this invention comprising both a compound of the invention and an additional therapeutic agent to a subject does not preclude the separate administration of said therapeutic agent, any other therapeutic agent or any compound of this invention to said subject at another time during a course of treatment.

Effective amounts of the other therapeutic agents are well known to those skilled in the art and guidance for dosing may be found in patents referenced herein. However, it is well within the skilled artisan's purview to determine the other therapeutic agent's optimal effective-amount range. In one embodiment of the invention where another therapeutic agent is administered to an animal, the effective amount of the compound of this invention is less than its effective amount would be where the other therapeutic agent is not administered. In another embodiment, the effective amount of the conventional agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

Additional therapeutic agents useful in the method of treatment are the same as those described above as part of combination compositions.

The compounds of this invention may be assayed in vitro by known methods. For instance, binding assays for the norepinephrine transporter in rat forebrain membranes or recombinant human transporter protein are available commercially, by MDS Pharma Services and NovaScreen Bioscience Corporation among others, and may be measured by known methods: see, e.g., Raisman R et. al. Eur. J. Pharmacol. 1982 78: 345; Langer S Z et. al. Eur. J. Pharmacol. 1981 72: 423. Ex vivo measurements on norepinephrine uptake can also be readily carried out; see e.g. Hyttel J, Prog. Neuropharmacol. Biol. Psychiatry 1982 6: 177.

Norepinephrine uptake inhibitors, including Compound 1, demonstrate the ability to reduce motor hyperactivity in an animal model of ADHD; see: Moran-Gates T et. al., Int. J. Neuropsychopharmacol. 2005 (Epub)[?]. Norepinephrine uptake inhibitors have also been demonstrated to decrease food intake in rats, analogous to the clinically observed anti-obesity effect in humans. Wong D T et. al., Neuropsychopharmacology 1993 8:23; Gehlert D R et. al. J Pharm. Exp. Ther. 1998 287: 122; Molloy B B and Schmiegel K K, U.S. Pat. No. 4,626,549 to Eli Lilly.

Each of the compounds of this invention may be tested for activity in such in vitro assays animal models. The compounds of the invention may also be tested in in vitro assays by means known in the art, to quantify resistance to liver metabolism compared to the corresponding non-deuterated, non-¹³C compound and therapeutic usefulness. See for instance Yan Z and Caldwell G W, Curr. Top. Med. Chem. 2001 5: 403; Rushmore T H, Kong A N, Curr. Drug Metab. 2002 3: 481; and references cited in these publications.

Diagnostic Methods and Kits

According to another embodiment, the invention provides a method of determining the concentration of Compound 1 in a biological sample, said method comprising the steps of:

a) adding a known concentration of a second compound to said biological sample, said second compound having the formula: 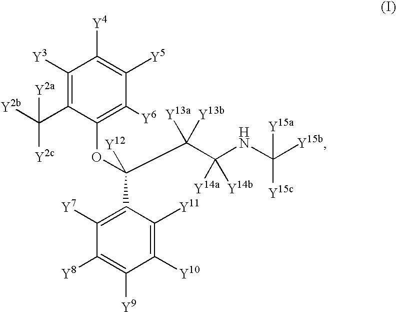
wherein at least one Y is deuterium or at least one carbon is replaced with ¹³C;

b) subjecting said biological sample to a measuring device that distinguishes Compound 1 from said second compound;

c) calibrating said measuring device to correlate the detected quantity of Compound 1 with the known concentration of said second compound added to said biological sample; and

d) determining the concentration of said compound in said biological sample by comparing the detected quantity of Compound 1 with the detected quantity and known concentration of said second compound.

Measuring devices that can distinguish Compound 1 from said second compound include any measuring device that can distinguish between two compounds that are of identical structure except that one contains one or more deuterium in place of one or more hydrogen, or one or more ¹³C in place of one or more ¹²C. Preferably, such a measuring device is a mass spectrometer.

In a preferred embodiment, at least three combined Y atoms and carbons are deuterium and ¹³C in said second compound; i.e. (total number of D)+(number of ¹³C)≧3.

In another preferred embodiment, the method comprises the additional step of organically extracting Compound 1 and said second compound from said biological sample prior to step b).

Compound 1 and the second compounds will have similar solubility, extraction, and chromatographic properties, but significantly different molecular mass. Thus, the second compound is useful as an internal standard in a method that comprises the step of organic extraction to measure the efficiency of that extraction and to ensure an accurate determination of the true concentration of Compound 1 (see Tuchman M and McCann M T, Clin. Chem. 1999 45: 571; Leis H J et. al., J. Mass Spectrom. 2001 36: 923; Taylor R L et. al. Clin. Chem. 2002 48: 1511, the disclosures of which are herein incorporated by reference).

The compounds of the present invention (the second compound) are particularly useful in this method since they are not radioactive and therefore do not pose a hazard to personnel handling the compounds. Thus, these methods do not require precautions beyond those normally applied in clinical sample analysis. Furthermore, stably labeled isotopes have long been used to assisting in research into the enzymatic mechanism of cytochrome P450 enzymes (Korzekwa K R et. al., Drug Metab. Rev. 1995 27: 45; Kraus, J A and Guengerich, F P, J. Biol. Chem. 2005 280: 19496; Mitchell K H et. al., Proc. Natl. Acad. Sci. USA 2003 109: 3784).

In another embodiment, a compound of this invention or its acid addition salt is exposed to a potentially metabolizing protein or biological mixture, for instance, an oxidative enzyme such as a cytochrome P450; or a liver microsome fraction or a liver slice. Reaction is monitored, for instance by following the disappearance of starting material or appearance of product, or both, using analytical methods known in the art, such as reversed-phase HPLC with UV absorption or mass spectroscopic detection. Concentrations of both enzyme and substrate (the compound of this invention) may be varied to determine kinetic parameters, for instance, by using appropriate nonlinear regression software such as is known in the art. In a separate experiment, the light isotopologue of the tested compound is tested by like means, yielding the separate kinetic parameters for comparison, as well if desired as an apparent steady-state deuterium isotope effect as ^{D}(V/K) determined as the ratio of products formed in the hydrogen versus deuterium reactions. Alternatively, a compound of this invention may be admixed with its light atom isotopologue in a competition experiment to determine rates of disappearance of the two compounds, making use of analytical instrumentation capable of differentiating between the two compounds based on their mass differences. Alternatively, or in addition, pre-steady state kinetics such as V₀ may be determined by means known in the art, for instance, using quench-flow apparatus, monitoring the quenched reactions at varying times after admixing reactive components.

In a related embodiment, the invention provides a diagnostic kit comprising a diagnostic compound having the formula I, wherein each Y is independently H or D: 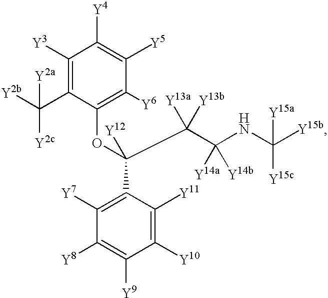
or an acid addition salt thereof, as described above, in a sealed vessel; and instructions for using said compound to determine the concentration of a test compound in a biological sample. In a preferred embodiment, at least three Y moieties in said diagnostic compound are deuterium.

In another embodiment, the invention provides a method of evaluating the metabolic stability of a compound of formula I, comprising the steps of contacting the compound of formula I or its acid addition salt with a metabolizing enzyme source for a period of time; and comparing the amount of said compound and metabolic products of said compounds after said period of time.

In one preferred embodiment, the method comprises an additional step of comparing the amount of said compound and said metabolic products of said compounds at an interval during said period of time. This method allows the determination of a rate of metabolism of said compound.

In another preferred embodiment, the method comprises the additional steps of contacting an isotopologue of said compound with said metabolizing enzyme source; comparing the amount of said isotopologue and metabolic products of said isotopologue after said period of time determining a rate of metabolism of said isotopologue; and comparing the metabolic stability of said compound and said isotopologue. This method is useful in determining at which sites on a compound of formula I a deuterium or ¹³C would cause the greatest increase in metabolic stability. It is also useful in comparing the metabolic stability of a compound of formula I with the metabolic stability of Compound 1.

A metabolizing enzyme source may be a purified, isolated or partially purified metabolic protein, such as a cytochrome P450; a biological fraction, such as a liver microsome fraction; or a piece of a metabolizing organ, such as a liver slice.

The determination of the amount of compound and its metabolic products is well known in the art. It is typically achieved by removing an aliquot from the reaction mixture and subjecting it to an analysis capable of distinguishing between the compound and its metabolites, such as reversed-phase HPLC with UV absorption or mass spectroscopic detection. Concentrations of both the metabolizing enzyme and the compound may be varied to determine kinetic parameters, for instance, by using appropriate nonlinear regression software such as is known in the art. By comparing the kinetic parameters of both a compound of formula I and Compound 1 an apparent steady-state deuterium isotope effect (^{D}(V/K)) can be determined as the ratio of products formed in the hydrogen versus deuterium reactions.

The determination of a rate of metabolism of an isotopologue may be achieved in a reaction separate from the reaction for determining the metabolism rate of the compound. Alternatively, the compound be admixed with an isotopologue in a competition experiment to determine rates of disappearance of the two compounds, making use of analytical instrumentation capable of differentiating between the two compounds based on their mass differences.

In yet another embodiment, pre-steady state kinetics, such as V₀, may be determined by means known in the art, for instance, using quench-flow apparatus, by monitoring the quenched reactions at varying times after mixing the compound or isotopologue with the metabolizing enzyme source.

In a related embodiment, the invention provides a kit comprising, in separate vessels: a) Compound 1; and b) a metabolizing enzyme source. The kit is useful for comparing the metabolic stability of a compound of formula I with Compound 1, as well as evaluating the affect of deuterium and ¹³C replacement at various positions on a compound of Formula I. In a preferred embodiment, the kit further comprises instructions for using Compound 1 and said metabolizing enzyme source to evaluate the metabolic stability of a compound of formula I.

In order that the invention might be more fully understood, the following examples are set forth. They are not intended to limit the scope of the invention and further examples will be evident to those of ordinary skill in the art. In each example set forth herein, carbon shall be ¹²C incorporated at its natural abundance unless otherwise specified.

### EXAMPLE 1

### 4-Deutero-1-methoxy-2-methylbenzene

4-Bromo-2-methylanisole (0.48 mol) is dissolved in 500 mL of dry THF and cooled to −78° C. under argon. The mixture is treated via cannula with 240 mL of n-butyllithium as a 2.1 M solution in hexanes. The mixture is stirred for 10 min in the cold, then is cannulated into a mixture of deuterium oxide (1.5 mol) and THF (300 mL), precooled to −40° C. After stirring for 15 min, the cooling bath is removed and the mixture is stirred for an additional 3 h, then concentrated in vacuo to about 200 mL. The residue is partitioned between 500 mL each ether and water. The organic layer is washed with 300 mL of brine, dried over MgSO₄, and concentrated in vacuo, and used without subsequent purification.

### EXAMPLE 2

### 4-Deutero-2-methylphenol (Formula XII, wherein Y⁴ is deuterium and all other Y substituents are hydrogen), Method A

A solution of the product of Example 1 (64 mmol) in 70 mL of methylene chloride is cooled to −78° C. and treated dropwise with 68 mmol of boron tribromide. The mixture is stirred for 1.5 h, then the CO₂/acetone bath is replaced with ice and the mixture stirred for an additional 5 h. The reaction is quenched with 50 mL of 1 N NaHCO₃ and the mixture partitioned between 150 mL of ether and an additional 50 mL of 1 N NaHCO₃. The organic layer is washed with brine, dried over MgSO₄, and concentrated in vacuo. Recrystallization from hexanes yields the title product.

### EXAMPLE 3

### 4-Deutero-2-methylphenol (Formula XII, wherein Y⁴ is deuterium and all other Y substituents are hydrogen), Method B

Deuteroiodic acid is prepared according to the method of Vogel A I, A textbook of Practical Organic Chemistry, 3rd Ed.; John Wiley: 1956 except using deuterium oxide in place of water and deuterium sulfide (generated from ZnS and 6 N deuterium chloride) in place of hydrogen sulfide. A suspension of 0.2 mol of 4-bromo-2-methylphenol (Fluorochem, Derbyshire UK) in 40 mL of 41% deuteroiodic acid is heated under reflux under an argon atmosphere for 17 h. After cooling, the mixture is partitioned between 350 mL each methylene chloride and saturated aqueous sodium thiosulfate. The organic layer is washed again with saturated aqueous sodium thiosulfate, then with with brine, dried over MgSO₄, and concentrated in vacuo. Recrystallization from hexanes, decolorizing with activated charcoal, yields the title product.

### EXAMPLE 4

### 4,6-Dideutero-2-methylphenol (Formula XII, wherein Y⁴ and Y⁶ are deuterium and all other Y substituents are hydrogen)

2,4-Dibromo-6-methylphenol (126 mmol; Gates P S et. al. U.S. Pat. No. 4,263,037 to Fisons) is reacted with deuteroiodic acid (28 mL) as set forth in the general procedure outlined in Example 3. Recrystallization from hexanes, decolorizing with activated charcoal, yields the title product.

### EXAMPLE 5

### 5-Deutero-1-methoxy-2-methylbenzene

17 mmol of 270 mesh magnesium powder is suspended in 2.5 mL of dry THF under argon and treated with 0.56 mmol of ethyl bromide. The mixture is stirred for 3 h, then an additional 2.5 mL of THF is added, followed by 0.53 mmol of anhydrous ferrous chloride and 0.6 mL of a 0.5 M magnesium chloride/THF solution. After an additional 3 min, 10.2 mmol of 5-chloro-2-methylanisole (ProSynth Ltd, Acton, UK) is added at ambient temperature during about 1 h with vigorous stirring. The mixture is stirred for 17 h, then cooled to −40° C. and treated via syringe with a solution of 15 mmol of deuteroacetic acid (CH₃CO₂D) in THF (4 mL). The mixture is stirred for 5 min, then evaporated in vacuo. The residue is suspended in toluene, filtered, and the filtrate evaporated. Silica gel flash chromatography (EtOAc/hexanes eluant) yields the title product.

### EXAMPLE 6

### 5-Deutero-2-methylphenol (Formula XII, wherein Y⁵ is deuterium and all other Y substituents are hydrogen)

A portion of the product of Example 5 (5.2 mmol) is demethylated as set forth in the general procedure outlined in Example 2. Recrystallization from hexanes yields the title product.

### EXAMPLE 7

### 2-(Deuteromethyl)-1-methoxybenzene

o-Cresol (14 mmol) is dissolved in 30 mL of carbontetrachloride and treated with 15 mmol of N-bromosuccinimide and 0.6 mmol of azobisisobutyronitrile (AIBN). The mixture is heated under refux for 3 h, then cooled to ambient temperature, filtered through diatomaceous earth and concentrated in vacuo. Silica gel flash chromatography (EtOAc/hexanes eluant) yields the title product.

### EXAMPLE 8

### 2-(Deutomethyl)phenol (Formula XII, wherein one of Y² are deuterium, two of Y² are hydrogen, and all other Y substituents are hydrogen)

A sample of the product of Example 7 (8.3 mmol) is lithiated and quenched with deuterium oxide according to the general procedure outlined in Example 1. Recrystallization from hexanes yields the title product.

### EXAMPLE 9

### tert-Butyldimethylsilyl-5-deutero-2-methoxybenzoate

A solution of 58 mmol of 5-bromo-2-methoxybenzoic acid (Auerback J et. al. U.S. Pat. No. 5,248,817 to Merck) and 60 mmol of diisopropylethylamine in 100 mL of methylene chloride is cooled in ice/water under argon and treated with 58 mmol of tert-butyldimethylsilyl chloride. The mixture is stirred for 15 h, warming slowly to ambient temperature, then is diluted with 200 mL of ether and washed with water and brine, dried over MgSO₄, and concentrated in vacuo. Silica gel flash chromatography (EtOAc/hexanes eluant) yields the title product.

### EXAMPLE 10

### Methyl-5-deutero-2-methoxybenzoate

A sample of the product of Example 9 (45 mmol) is lithiated and quenched with deuterium oxide according to the general procedure outlined in Example 1. The product thus obtained is dissolved in 60 mL of dry methanol, cooled in an ice/water bath, and treated with 12.5 mL of anhydrous 4N HCl in dioxane. The cold bath is removed and the solution is stirred for 6 h, then concentrated in vacuo. Silica gel flash chromatography (EtOAc/hexanes eluant) yields the title product.

### EXAMPLE 11

### (5-Deutero-2-methoxyphenyl)-dideuteromethanol

A solution of the product of Example 10 (32 mmol) in 40 mL of dry THF is cooled in a CO₂/acetone bath. A −78° C. solution of 32 mL of 1 N lithium aluminum deuteride in THF (Aldrich Chemicals) is cannulated into the solution of the ester during about 30 min and the mixture is stirred for 16 h, warming slowly to room temperature. The solution is cooled in ice/water and treated cautiously dropwise, sequentially, with 1.4 mL of water, 1.4 mL of 15% aqueous NaOH, and 4.2 mL of water. The mixture is filtered through Celite, dried over MgSO₄, and concentrated in vacuo. Silica gel flash chromatography (EtOAc/hexanes eluant) yields the title product.

### EXAMPLE 12

### 4-Deutero-2-(bromodideuteromethyl)-1-methoxybenzene

A solution of product of Example 11 (23 mmol) in 40 mL of dichloromethane is cooled in an ice/water bath under argon and treated with 25 mmol each of triphenylphosphine and carbon tetrabromide. The ice bath is removed and the mixture allowed to react for 4 h and then concentrated in vacuo. The residue is triturated with 2:1 ethyl acetate/hexane and filtered, and the filtrated concentrated in vacuo. Silica gel flash chromatography (EtOAc/hexanes eluant) yields the title product.

### EXAMPLE 13

### 4-Deutero-2-(trideuteromethyl)-1-methoxybenzene

A 18 mmol portion of product of Example 12 is lithiated and quenched with deuterium oxide according to the general procedure outlined in Example 1. Silica gel flash chromatography (EtOAc/hexanes eluant) yields the title product.

### EXAMPLE 14

### 4-Deutero-2-(trideuteromethyl)-phenol (Formula XII, wherein each of Y² is deuterium, Y⁴ is deuterium, and all other Y substituents are hydrogen)

A 14 mmol portion of product of Example 13 is demethylated as set forth in the general procedure outlined in Example 2. Recrystallization from hexanes yields the title product.

### EXAMPLE 15

### (E)-(¹³C₆-phenyl)prop-2-en-1-ol (Formula XV, wherein all Y substituents are hydrogen and each carbon atom in the phenyl ring is ¹³C)

A solution of 3.5 mmol allyl alcohol in 3.5 mL of acetonitrile is argon/vacuum degassed (3×). To it is added, sequentially under argon, 70 mmol of freshly ground potassium carbonate powder, 0.105 mmol of tri-o-tolylphosphine, 0.035 mmol of palladium acetate, and 3.5 mmol of bromobenzene-¹³C₆ (Aldrich Chemicals). The mixture is heated under reflux for 16 h, and partitioned between 25 mL each ether and water. The aqueous layer is washed with additional ether, and the combined organic layers are washed with brine, dried over MgSO₄, and concentrated in vacuo. Silica gel flash chromatography (methanol/methylene chloride eluant) yields the title product.

### EXAMPLE 16

### ((2R,3R)-3-(¹³C₆-phenyl)oxiran-2-yl)methanol

A flame-dried flask is charged with 0.47 mmol of D-(−)-diisopropyl tartrate and 60 mL of methylene chloride and cooled to −20° C. under argon. The mixture is treated sequentially with 350 mg of powdered activated 4A molecular sieves, 0.32 mmol of titanium tetra-isopropoxide, and 12.5 mmol of tert-butyl hydroperoxide as an 8.1 M solution in methylene chloride, and is then stirred for 1 h in the cold. To this suspension is added, dropwise during 1 h, 6.2 mmol of the product of Example 15 as a solution in 1.2 mL of methylene chloride using a syringe pump. The mixture is stirred for 17 h at −20° C., then treated with 0.12 mL of 10% aq. NaOH solution saturated with NaCl, followed by 6 mL of ether. The solution is allowed to warm to about 10° C. and after about 10 min at that temperature, 135 mg of MgSO₄ and about 40 mg of Celite are added. The mixture is stirred for several minutes, then allowed to stand and the supernatant is filtered through Celite, washing with additional ether. The filtrate is concentrated in vacuo and the residue is treated with about 20 mL of toluene and again concentrated in vacuo. Recrystallization (ether/petroleum ether) yields the title compound.

### EXAMPLE 17

### (S)-1-(¹³C₆-phenyl)propane-1,3-diol

A solution of 4.1 mmol of the product of Example 16 in 20 mL of dimethoxyethane is cooled in an ice/water bath under argon and treated dropwise with 4.3 mmol of a 4.0 M solution of sodium bis(2-methoxyethoxy)aluminum hydride in toluene. After removing the ice bath the reaction proceeds for 5 h at room temperature. To the mixture is added 80 mL of ether and 30 mL of 10% aqueous potassium sodium (+) tartrate tetrahydrate (first few mL dropwise). The aqueous layer is extrated with additional ether and the combined organics are washed with brine, dried over MgSO₄, and concentrated in vacuo to yield the oily product which is used for subsequent reaction without purification.

### EXAMPLE 18

### (S)-3-Hydroxy-3-(¹³C₆-phenyl)propyl methanesulfonate (Formula X wherein X is methanesulfonate, each Y is hydrogen, Z is hydroxyl, and each carbon in the phenyl ring is ¹³C)

A solution of the entire yield of Example 17 (holding back a ca. 2 mg sample) is dissolved in 25 mL of methylene chloride, treated with 5.0 mmol of diisopropylethylamine, and cooled under argon at −10° C. Methane sulfonyl chloride (4.5 mmol) is added dropwise during several minutes and the mixture is stirred for 2.5 h in the cold. The mixture is diluted with 15 mL of ether and washed sequentially with 10% KHSO₄, saturated sodium bicarbonate solution, and brine, then dried over MgSO₄, and concentrated in vacuo. Silica gel flash chromatography (ethyl acetate/hexanes eluant) yields the title product.

### EXAMPLE 19

### (R)-3-(4-Deutero-2-methylphenoxy)-3-(¹³C₆-phenyl)propyl methanesulfonate (Formula XIII wherein X is methanesulfonate, Y⁴ is deuterium and all other Y substituents are hydrogen, and each carbon in the phenyl ring is ¹³C)

A solution of 2.4 mmol of the product of Example 18 in 25 mL of ether is treated with 4.8 mmol of 4-deutero-2-methylphenol, and 3.6 mmol of triphenylphosphine. The mixture is cooled in an ice/methanol bath and 3.6 mmol diethyl azodicarboxylate is added dropwise during several minutes. The mixture is stirred in the cold for 4 h, then concentrated in vacuo. The residue is triturated with 1:2 ethyl acetate/hexanes, filtered through Celite, and the filtrate is concentrated and purified by silica gel flash chromatography (ethyl acetate/hexanes eluant) to yield the title product.

### EXAMPLE 20

### (R)-3-(4-Deutero-2-methylphenoxy)-N-(methyl)-3-(¹³C₆-phenyl)propan-1-amine hydrochloride (Compound 2 wherein each carbon in the unmethylated phenyl ring is ¹³C)

A solution of 0.8 mmol the product of Example 19 in 5 mL of THF is treated with 5 mL of 40% methylamine in water. The mixture is heated for 3 h at 65° C., then partitioned between 30 mL each ether and saturated sodium bicarbonate solution. The organic layer is washed with brine, then dried over MgSO₄ and concentrated in vacuo. The residue is dissolved in 10 mL of ether and treated, under argon, with HCl gas to form a white precipitate. The salt is recrystallized from acetonitrile to yield the title compound.

### EXAMPLE 21

### (R)-3-(4-Deutero-2-methylphenoxy)-3 (¹³C₆-phenyl)-N-(trideuteromethyl)-propan-1-amine hydrochloride (Compound 105 wherein each carbon in the unmethylated phenyl ring is ¹³C)

A solution of 0.5 mmol the product of Example 19 is dissolved in 2 mL of DMS0 in a pressure vial and treated with 5 mmol of methyl-d₃-amine hydrochloride (Aldrich Chemicals) and 5 mmol of diisopropylethylamine. The mixture is capped and heated at 70° C. for 15 h, then partitioned between 25 mL each of ether and saturated sodium bicarbonate solution. The aqueous layer is extracted with more ether and the combined organic layers are washed with brine, dried over MgSO₄ and concentrated in vacuo. The residue is dissolved in 10 mL of ether and treated, under argon, with HCl gas to form a white precipitate. The salt is recrystallized from acetonitrile to yield the title compound.

### EXAMPLE 22

### (R)-3-(4-Deutero-2-methylphenoxy)-3-(¹³C₆- phenyl)-N-(¹³C-trideuteromethyl)-propan-1-amine hydrochloride (Compound 105 wherein each carbon in the unmethylated phenyl ring is ¹³C and the carbon of the N-methyl group is ¹³C

A 0.5 mmol portion of the product of Example 19 reacted with 5 mmol of methyl-¹³C d₃-amine hydrochloride (Aldrich Chemicals) using the general procedure set forth in Example 21. The salt is recrystallized from acetonitrile to yield the title compound.

### EXAMPLE 23

### (E)-3-(pentadeuterophenyl)prop-2-en-1-ol (Formula XV, wherein Y⁷⁻¹¹ are deuterium and all other Y substituents are hydrogen)

To a solution of 11 mmol of trans-cinnamic-d₅ acid-OD (C₆D₅CH═CHCO₂D; C/D/N Isotopes) in 25 mL of dry THF, cooled in an ice/water bath under argon, is added 22 mL of 1M BH₃THF in THF during 15 min. The mixture is stirred overnight, warming to room temperature, and then cooled again in ice/water and quenched with 1 N HCl. The reaction is evaporated to a small volume and the residue partitioned between 40 mL each ethyl acetate and 10% KHSO4 solution. The organic layer is washed with brine, dried over MgSO₄, and concentrated in vacuo. Silica gel flash chromatography (methanol/methylene chloride eluant) yields the title product.

### EXAMPLE 24

### ((2R,3R)-3-(pentadeuterophenyl)oxiran-2-yl)methanol

A 9 mmol sample of the product of Example 23 is epoxidized using the general procedure set forth in Example 16.

### EXAMPLE 25

### (S)-1-(pentadeuterophenyl)propane-1,3-diol

Regioselective reduction of a 6.5 mmol sample of of the product of Example 24 is carried out using the general procedure set forth in Example 17. The product is used for the subsequent step without purification.

### EXAMPLE 26

### (S)-3-Hydroxy-3-(pentadeuterophenyl)propyl methanesulfonate (Formula X wherein X is methanesulfonate, Y⁷⁻¹¹ are deuterium and all other Y substituents are hydrogen, and Z is hydroxyl)

Reaction of 5.2 mmol sample of the product of Example 25 with methanesulfonyl chloride is carried out using the general procedure set forth in Example 18. Silica gel flash chromatography (ethyl acetate/hexanes eluant) yields the title product.

### EXAMPLE 27

### (R)-3-(Pentadeuterophenyl)-3-(4,6-dideutero-2-methylphenoxy)propyl methanesulfonate (Formula XIII wherein X is methanesulfonate, Y⁴ and Y⁶⁻¹¹ are deuterium and all other Y substituents are hydrogen)

Reaction of a 1.8 mmol portion of the product of Example 26 with a 1.8 mmol portion of the product of Example 4 is carried out using the general procedure set forth in Example 19. Silica gel flash chromatography (ethyl acetate/hexanes eluant) yields the title product.

### EXAMPLE 28

### (R)-N-Methyl-3-(pentadeuterophenyl)-3-(4,6-dideutero-2-methylphenoxy)propan-1-amine hydrochloride (Compound 332)

Reaction of a 0.4 mmol sample of the product of Example 27 with methylamine, followed by hydrogen chloride, is carried out using the general procedure set forth in Example 20. Recrystallization of the resulting hydrchloride salt yields the title product.

### EXAMPLE 29

### (R)-3-(4,6-Dideutero-2-methylphenoxy)-3-(pentadeuterophenyl)-N-(trideuteromethyl)-propan-1-amine hydrochloride (Compound 556)

Reaction of a 0.4 mmol sample of the product of Example 27 with trideuteromethylamine, followed by hydrogen chloride, is carried out using the general procedure set forth in Example 21. Recrystallization of the resulting hydrchloride salt yields the title product.

### EXAMPLE 30

### (S)-3-(Phenyl)-3-trimethylsilyloxy)propanenitrile

A solution of 8.6 mmol of (R)-3-hydroxy-3phenylpropanenitrile (Kumar P and Pandley R K, U.S. Pat. No. 6,838,581 to Council of Scientific and Industrial Research) and 9.5 mmol of diisopropylethylamine in 20 mL of methylene chloride is cooled in an ice/methanol bath under argon. To it is added 9.5 mmol of trimethylsilyl chloride dropwise during several minutes. The mixture is stirred for 15 min in the cold, the ice bath is removed and stirring is continued for 1.5 h. The mixture is concentrated and the residue is partitioned between ether and brine. The organic layer is dried over MgSO₄, and concentrated in vacuo, and the title product is used without subsequent purification.

### EXAMPLE 31

### (S)-3-(Amino)-3,3-dideutero-1-phenylpropan-1-ol

A suspension of 17.2 mmol of lithium aluminum deuteride (Aldrich chemicals) in 20 mL of ether is cooled in an ice/methanol bath under argon with a solution of the entire product of Example 30, save about 2 mg, in 10 mL of ether. The mixture is stirred for 10 min in the cold and the ice bath is removed and stirring is continued for 17 h. After cooling in ice/water, the excess reducing agent is quenched by careful dropwise addition of 0.65 mL of water, 0.65 mL of 15% aqueous NaOH, and 1.95 mL of water. The mixture is filtered and the filter cake washed well with ether. The organic layer is dried over MgSO₄, and concentrated in vacuo, and the title product is used without subsequent purification.

### Example 32

### (S)-3,3-Dideutero-1-phenyl-3-(dimethylmino)-propan-1-ol

One half of the product of Example 31 is dissolved in 20 mL of methanol, cooled in an ice/water bath, and treated with 2.5 mL of formalin as a 37% solution in water, followed by 42 mmol of sodium cyanoborohydride. The ice bath is removed and the pH is adjusted to about 6 (wet pH paper) with glacial acetic acid. The mixture is stirred for 18 h, concentrated in vacuo, and the residue is partitioned between ether and 1 N NaOH. The aquous layer is extracted with ether and the combined organic layers are dried over MgSO₄ and concentrated in vacuo. Silica gel flash chromatography (methanol/methylene chloride/saturated aq. ammonium hydroxide eluant) yields the title product.

### EXAMPLE 33

### (R)-3 -(5-Deutero-2-methylphenoxy)-1,1-dideutero-N,N-dimethyl-3-phenylpropan-1-amine

Reaction of a 1.4 mmol portion of the product of Example 32 with a 1.4 mmol portion of the product of Example 6 is carried out using the general procedure set forth in Example 19. Silica gel flash chromatography (methanol/methylene chloride eluant) yields the title product.

### EXAMPLE 34

### (R)-3-(5-Deutero-2-methylphenoxy)-1,1-dideutero-N-methyl-3-phenylpropan-1-amine

A solution of 1.1 mmol of the product of Example 33 in 3 mL of toluene is heated to 55° C. Then 1.3 mmol of diisopropylethylamine is added, followed by the dropwise addition of 1.6 mmol of phenyl chloroformate. The mixture is stirred at 55° C. for 1.5 h, and 4 ml of 1 N sodium bicarbonate solution is added. The mixture is stirred for ten minutes at about 45° C., and the phases are separated. The organic phase is washed twice with 0.5N hydrochloric acid, and then washed with 1 N sodium bicarbonate solution. The organic phase is dried over MgSO₄, evaporated under vacuum, and the residue is taken up in 5 ml of dimethylsulfoxide. The mixture is heated to 45° C. and 5 mmol of sodium hydroxide and 6 ml of water is added dropwise. The basic mixture is stirred for about 18 hours at 50° C., diluted with 3.5 ml of water, and acidified to pH 5.0-5.5 by addition of acetic acid. Then 4 ml of hexane is added, the mixture is stirred for ten minutes, and the phases are separated. The aqueous phase is basified to pH ˜11 by addition of 4N aqueous sodium hydroxide, and 3 ml of ethyl acetate is added. After stirring for 15 minutes, the phases are separated, and the aqueous layer is extracted with ethyl acetate. The combined organic extracts are washed with brine and concentrated in vacuo. The residue is taken up in dry ether and treated under argon with a slow stream of anhydrous hydrogen chloride gas to form a white precipitate. The solids are filtered, washed with ether, and dried in vacuo to yield the title product.

### EXAMPLE 35

### (R)-3-(2-Methyl-3,4,5,6-tetradeuterophenoxy)-3-phenylpropyl methanesulfonate (Formula XIII wherein X is methanesulfonate, Y³⁻⁶ is deuterium and all other Y substituents are hydrogen)

A solution of 6.3 mmol of o-cresol-3,4,5,6-d₄ OD (CH₃C₆D₄OD; C/D/N Isotopes) is reacted with 6.3 mmol of (S)-3-hydroxy-3-phenylpropyl methanesulfonate (Gao Y and Sharplesss K B, J. Org. Chem. 1988 53: 4081) using the general procedure set forth in Example 19. Silica gel flash chromatography (ethyl acetate/hexanes eluant) yields the title product.

### EXAMPLE 36

### (R)-N-Methyl-3-(2-methyl-3,4,5,6-tetradeuterophenoxy)-3-phenylpropan-1-amine hydrochloride (Compound 16)

A 2.9 mmol portion of product of Example 35 is reacted with methylamine, followed by hydrogen chloride, using the general procedure set forth in Example 20.

Recrystallization of the resulting hydrochloride salt from acetonitrile/diisopropyl ether yields the title product.

### EXAMPLE 37

### (R)-3-Phenyl-3-(2-trideuteromethyl-3,4,5,6-tetradeuterophenoxy)propyl methanesulfonate (Formula XIII wherein X is methanesulfonate, each Y² is deuterium, Y³⁻⁶ are deuterium, and all other Y substituents are hydrogen)

A 3.3 mmol sample of o-cresol-d₇ (CD₃C₆D₄0H; C/D/N Isotopes) is reacted with 6.3 mmol of (S)-3-hydroxy-3-phenylpropyl methanesulfonate using the general procedure set forth in Example 19. Silica gel flash chromatography (ethyl acetate/hexanes eluant) yields the title product.

### EXAMPLE 38

### (R)-N-Methyl-3-phenyl-3-(2-trideuteromethyl-3,4,5,6-tetradeuterophenoxy)-propan-1-amine hydrochloride (Compound 40)

A 2.7 mmol sample of product of Example 37 is reacted with methylamine, followed by hydrogen chloride, using the general procedure set forth in Example 20.

Recrystallization of the resulting hydrochloride salt from acetonitrile/diisopropyl ether yields the title product.

### EXAMPLE 39

### (R)-(2-Deutermethyl)phenoxy-3-phenylpropyl methanesulfonate (Formula XIII wherein X is methanesulfonate, one of Y² is deuterium, and all other Y substituents are hydrogen)

A 1.3 mmol sample of the product of Example 8 is reacted with 1.3 mmol of (S)-3-hydroxy-3-phenylpropyl methanesulfonate using the general procedure set forth in Example 19. Silica gel flash chromatography (ethyl acetate/hexanes eluant) yields the title product.

### EXAMPLE 40

### (R)-3-(2-Deutermethyl)phenoxy-N-methyl-3-phenylpropan-1-amine hydrochloride (Compound 3)

A 2.7 mmol sample of product of Example 39 is reacted with methylamine, followed by hydrogen chloride, using the general procedure set forth in Example 20.

Recrystallization of the resulting hydrochloride salt from acetonitrile yields the title product.

### EXAMPLE 41

### (R)-3-(4-Deutero-2-(trideuteromethyl)phenoxy)-3-phenylpropyl methanesulfonate (Formula XIII wherein X is methanesulfonate, each Y² is deuterium, Y⁴ is deuterium, and all other Y substituents are hydrogen)

A 7.8 mmol sample of the product of Example 14 is reacted with 7.8 mmol of (S)-3-hydroxy-3-phenylpropyl methanesulfonate using the general procedure set forth in Example 19. Silica gel flash chromatography (ethyl acetate/hexanes eluant) yields the title product.

### EXAMPLE 42

### (R)-3-(4-Deutero-2-(trideuteromethyl)phenoxy)-N-methyl-3-phenylpropan-1-amine hydrochloride (Compound 33)

A 3.6 mmol sample of product of Example 41 is reacted with methylamine, followed by hydrogen chloride, using the general procedure set forth in Example 20.

Recrystallization of the resulting hydrochloride salt from acetonitrile/diisopropyl ether yields the title product.

### EXAMPLE 43

### (R)-3-(4-Deutero-2-(trideuteromethyl)phenoxy)-N-methyl-3-phenylpropan-1-amine hydrochloride (Compound 129)

A 2.0 mmol sample of product of Example 41 is reacted with trideuteromethylamine, followed by hydrogen chloride, using the general procedure set forth in Example 21.

Recrystallization of the resulting hydrochloride salt from acetonitrile/diisopropyl ether yields the title product.

### EXAMPLE 44

### (R)-3-(4,6-Dideutero-2-methylphenoxy)-3-phenylpropyl methanesulfonate (Formula XIII wherein X is methanesulfonate, Y⁴ is deuterium, Y⁶ is deuterium, and all other Y substituents are hydrogen)

A 6.4 mmol sample of the product of Example 4 is reacted with 6.4 mmol of (S)-3-hydroxy-3-phenylpropyl methanesulfonate using the general procedure set forth in Example 19. Silica gel flash chromatography (ethyl acetate/hexanes eluant) yields the title product.

### EXAMPLE 45

### (R)-3-(4,6-Dideutero)-2-methylphenoxy-N-methyl-3-phenylpropan-1-amine hydrochloride (Compound 12)

A 4.1 mmol sample of product of Example 44 is reacted with methylamine, followed by hydrogen chloride, using the general procedure set forth in Example 20.

Recrystallization of the resulting hydrochloride salt from acetonitrile yields the title product.

### EXAMPLE 44

### (R)-3-(4-Deutero-2-methylphenoxy)-3-phenylpropyl methanesulfonate (Formula XIII wherein X is methanesulfonate, each Y⁴ is deuterium and all other Y substituents are hydrogen)

A 16 mmol sample of 4-deutero-2-methylphenol is reacted with 16 mmol of (S)-3-hydroxy-3-phenylpropyl methanesulfonate using the general procedure set forth in Example 19. Silica gel flash chromatography (ethyl acetate/hexanes eluant) yields the title product.

### EXAMPLE 45

### (R)-3-(4-Deutero-2-methylphenoxy)-N-methyl-3-phenylpropan-1-amine hydrochloride (Compound 2)

A 9.4 mmol sample of product of Example 44 is reacted with methylamine, followed by hydrogen chloride, using the general procedure set forth in Example 20.

Recrystallization of the resulting hydrochloride salt from acetonitrile yields the title product.

### EXAMPLE 46

### (R)-3-(4-Deutero-2-methylphenoxy)-N-(¹³C-methyl)-3-phenylpropan-1-amine hydrochloride (Compound 2 wherein the N-methyl group is substituted with ¹³C)

A 9.4 mmol sample of product of Example 44 is reacted with ¹³C-methylamine hydrochloride (Aldrich Chemicals), followed by hydrogen chloride, using the general procedure set forth in Example 21. Recrystallization of the resulting hydrochloride salt from acetonitrile/diisopropyl ether yields the title product.

### EXAMPLE 47

### Binding to the norepinephrine transporter

Binding of test compounds to human recombinant norepinephrine transporter is conducted by displacement of [³H] nioxetine (NovaScreen; Hanover, Md.) using a modification of the method of Raisman R et. al., Eur. J. Pharmacol. 1982 78: 345. Compounds 2, 3, 12, 16, 33, 40, 129, 332, 556, 2 (wherein the non-methyl bearing phenyl ring is fully substituted with ¹³C), 105 (wherein the non-methyl bearing phenyl ring is fully substituted with ¹³C), and 105 (wherein each carbon in the unmethylated phenyl ring is ¹³C and the carbon of the N-methyl group is ¹³C) demonstrate dose-dependent displacement of [³H] nioxetine with an IC₅₀ of <5 μM.

### EXAMPLE 48

Ex-vivo inhibition of rat synaptosome norepinephrine transport. Inhibition of the accumulation of into rat whole brain synaptosomes and [³H]-norepinephrine into synaptosomes from rat frontal plus temporal cortex is measured essentially according to literature methods (Hyttel J, Prog. Neuropharmacol. Biol. Psychiatry 1982 6: 177). Briefly, rats are decapitated and the relevant brain tissue is rapidly removed and homogenized in 40 vol (w/v) ice-cold 0.32 M sucrose solution. The synaptosomal fraction (P2) is isolated by centrifugation at 600 g for 10 min, and the supernatant is centrifuged at 20,000 g for 55 min. The pellet (P2) is resuspended in modified Krebs-Ringer phosphate buffer [122 mM NaCl, 5 mM KCl, 972 mM CaCl₂, 1.2 mM MgSO₄, 10 mM glucose, 101 mM ascorbic acid, 161 mM ethylenediamine tetraacetic acid (EDTA), 16 mM phosphate buffer, pH 7.4]. [³H]-norepipnephrine (10 nM) is added and the samples are incubated with varying concentrations of a compound of this invention or a vehicle only control. The norephinephrine transporter assay is incubated at room temperature for 5 min. For all assays the incubation is terminated by rapid vacuum filtration using the assay buffer as filtration buffer. Background activities in the norephinephrine assays are defined as counts in the presence of 10 μM of talsupram. Each of the compounds will show active inhibition of norepinephrine uptake.

All references cited herein, whether in print, electronic, computer readable storage media or other form, are expressly incorporated by reference in their entirety, including but not limited to, abstracts, articles, journals, publications, texts, treatises, technical data sheets, internet web sites, databases, patents, patent applications, and patent publications. The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

In one embodiment, the compound or mixture of compounds is isolated. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

I claim:

1. A compound of formula I: 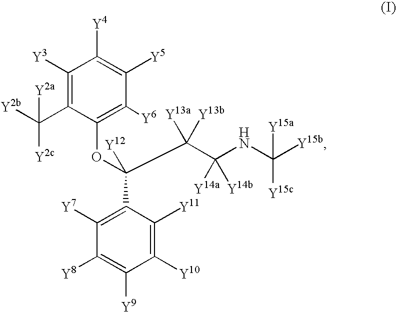
a prodrug thereof; or a pharmaceutically acceptable salt of said compound or prodrug; or a solvate, hydrate, and/or polymorph of said compound, salt, prodrug or prodrug salt, wherein:
at least one Y is deuterium, and the hydrogen attached to N is optionally deuterium, and each carbon is optionally replaced with ¹³C.

2. The compound according to claim 1, wherein the salt or prodrug salt is pharmaceutically acceptable.

3. The compound according to claim 2, wherein at least one of Y^{2a}, Y^{2b}, Y^{2c}, Y⁴, Y^{15a} , Y^{15b}, or Y^{15c} is deuterium.

4. The compound according to claim 3, wherein Y⁴ is deuterium.

5. The compound according to claim 4, wherein each of Y⁴, Y⁵, and Y⁶ is deuterium.

6. The compound according to claim 3, wherein one or more of Y^{15a}, Y^{15b}, or Y^{15c} is deuterium.

7. The compound according to claim 3, wherein one or more of Y^{2a}, Y^{2b}, or Y^{2c} is deuterium.

8. The compound according to claim 7, wherein one or more of Y^{15a}, Y^{15b}, or Y^{15c} is deuterium.

9. The compound according to claim 7, wherein each of Y^{2a}, Y^{2b}, and Y^{2c} is deuterium.

10. The compound according to claim 2, wherein said compound is a substantially isolated isostcreomer.

11. The compound according to claim 2, selected from: 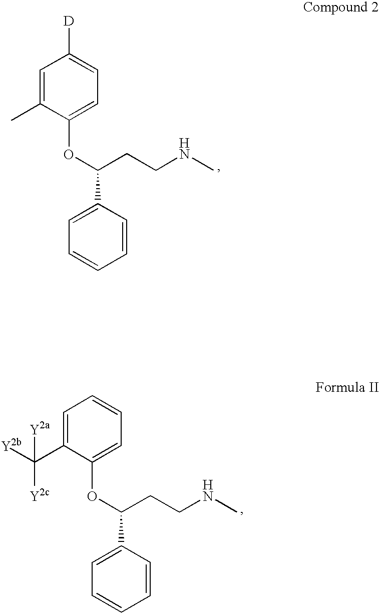 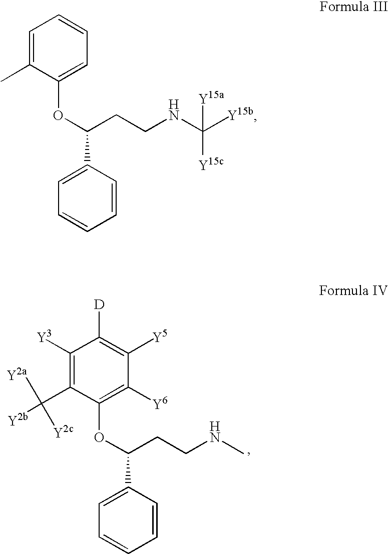 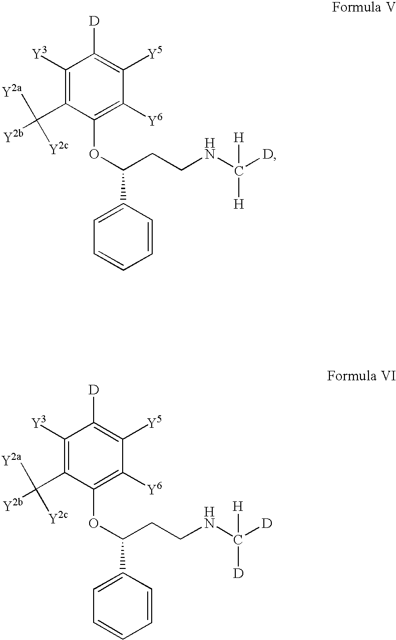 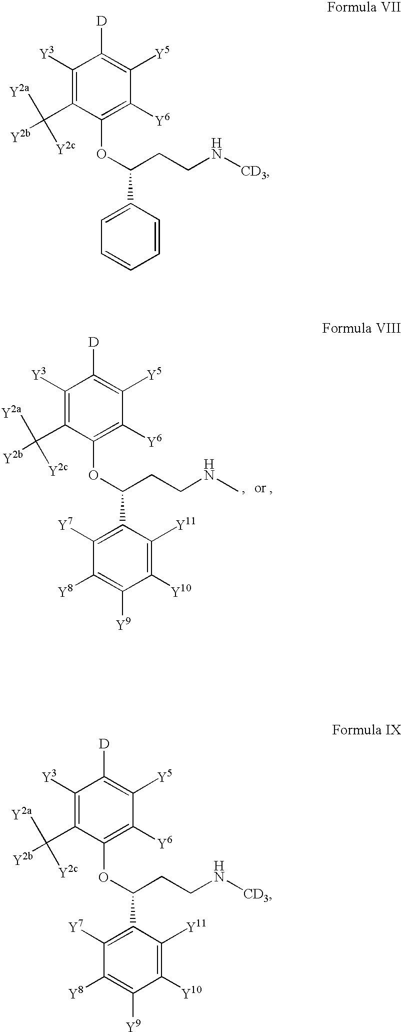
wherein in each compound the H attached to N Is optionally replaced with deuterium; and one or more carbons are optionally replaced by with ¹³C.

12. The compound according to claim 11, wherein said compound is selected from Compound 2 or any one of a compound of formula III, IV, VI, or VII.

13. The compound according to claim 12, wherein said compound is selected from any one of compound number 2, 13, 16, 21, 24, 29, 32, 37, 40, 109, 112, 117, 120, 125, 128, 133, or 136.

14. (canceled)

15. (canceled)

16. A mixture consisting essentially of:
a. a compound of formula I or an acid addition salt thereof; and
b. lighter isotopologues of said compound of formula I,
wherein at least 50% of said mixture is said compound of formula I.

17. A mixture consisting essentially of:
a. a compound of formula I or an acid addition salt thereof; and
b. lighter isotopologues of said compound of formula I,
wherein at least 50% of the compounds in said mixture comprise an isotope at each position occupied by an isotope in the compound of formula I.

18. A composition comprising an effective amount of a compound of formula I, or a pharmaceutically acceptable salt, solvate, hydrate, polymorph or prodrug thereof; and an acceptable carrier.

19. (canceled)

20. The composition according to claim 19 further comprising an effective amount of an additional therapeutic agent, wherein said additional therapeutic agent is useful for treating or preventing a condition selected from pain; inflammation;CNS disorders; ADHD; vasomotor symptoms; nervous system depression or anxiety; phobias; avoidant personality disorder; eating disorders; chemical dependencies; Parkinson's diseases; obsessive-compulsive disorder; negative symptoms of schizophrenia; premenstrual syndrome; headache; cognitive failure; obesity; functional bowel disorders; and major psychotic disorders.

21. (canceled)

22. (canceled)

23. (canceled)

24. A method of treating a subject suffering from or susceptible to ADHD; psoriasis; urinary incontinence; tic disorders; cognitive failure; chronic pain; stuttering or other communication disorders; pervasive developmental disorders; learning disabilities or motor skills disorders; anxiety disorders; cognitive disorders; or vasomotor symptoms; said method comprising the step of administering to said subject a composition comprising an effective amount of a compound of formula I, or a pharmaceutically acceptable salt solvate, hydrate, polymorph or prodrug thereof; and an acceptable carrier.

25. The method according to claim 24, wherein the subject is treated to alleviate or prevent attention-deficit/hyperactivity disorder.

26-43. (canceled)

44. A method of determining the concentration of Compound 1 in a biological sample comprising the steps of:
a, adding a known concentration of a compound of formula I, or an acid addition salt thereof, to a biological sample;
b. subjecting said biological sample to a measuring device that distinguishes Compound 1 from said compound of formula I;
c. calibrating said measuring device to correlate the detected quantity of said compound of formula I with the known concentration of said compound of formula I added to said biological sample; and
determining the concentration of Compound 1 in said biological sample by comparing the detected quantity of Compound 1 with the detected quantity and known concentration of said compound of formula I.

45-55. (canceled)
